(19) United States
(12) Patent Application Publication
(10) Pub. No.: US 2022/0121968 A1
(43) Pub. Date: Apr. 21, 2022

(54) FORECASTING AND LEARNING ACCURATE AND EFFICIENT TARGET POLICY PARAMETERS FOR DYNAMIC PROCESSES IN NON-STATIONARY ENVIRONMENTS

(71) Applicant: Adobe Inc., San Jose, CA (US)

(72) Inventors: Yash Chandak, Amherst, MA (US); Georgios Theocharous, San Jose, CA (US); Sridhar Mahadevan, Morgan Hill, CA (US)

(21) Appl. No.: 17/072,868

(22) Filed: Oct. 16, 2020

(51) Int. Cl. G06N 5/04; G06Q 10/06; G06Q 10/10

(52) U.S. Cl. CPC G06N 5/04; G06Q 30/0631; G06Q 10/10; G06Q 10/06375

(57) ABSTRACT

The present disclosure relates to systems, methods, and non-transitory computer-readable media that determine target policy parameters that enable target policies to provide improved future performance, even in circumstances where the underlying environment is non-stationary. For example, in one or more embodiments, the disclosed systems utilize counter-factual reasoning to estimate what the performance of the target policy would have been if implemented during past episodes of action-selection. Based on the estimates, the disclosed systems forecast a performance of the target policy for one or more future decision episodes. In some implementations, the disclosed systems further determine a performance gradient for the forecasted performance with respect to varying a target policy parameter for the target policy. In some cases, the disclosed systems use the performance gradient to efficiently modify the target policy parameter, without undergoing the computational expense of expressly modeling variations in underlying environmental functions.

### BACKGROUND

Recent years have seen significant advancement in hardware and software platforms for computer modeling and forecasting of various real-world environments. For example, many conventional systems model (e.g., using a Markov Decision Process) and forecast the state changes of an agent (e.g., a controller for a mechanical system or a digital system that interacts with another digital system, etc.) executing actions within a real-world environment. These systems can provide various benefits using the analyses provided by such computer-implemented models. To illustrate, conventional systems can generate digital recommendations to distribute digital content items across computer networks to client devices or modify its own parameters or the parameters of another computer-implemented system to improve performance.

Despite these advances however, conventional agent-environment modeling systems suffer from several technological shortcomings that result in inflexible, inaccurate, and inefficient operation of implementing computing devices. For example, conventional agent-environment modeling systems are often inflexible in their policy implementation. Indeed, conventional systems often implement policies that influence the action-selection decision of an agent when in a particular state, often with the aim of optimizing the resulting reward. Many conventional systems, however, implement fixed policies under the assumption that the environment (or the agent itself) is also fixed. But real-world, practical problems often involve several complex changing dynamics over time. Thus, conventional systems typically fail to flexibly adapt their policies to accommodate these changes.

In addition to flexibility concerns, conventional agent-environment modeling systems can also operate inaccurately and inefficiently. Indeed, by failing to flexibly adapt policies to changes in the environment, conventional agent-environment modeling systems are often inaccurate in that they fail to implement policies promoting decisions that lead to optimal rewards. Some conventional systems attempt to avoid these issues by modifying the current policies in response to changes to the environment, but these systems typically only do so after observing those changes, causing sub-optimal performance until the policy is updated. Other conventional systems implement methods that search for initial parameters that are effective despite changes over time. To illustrate, at least one conventional system utilizes meta-learning along with training tasks to find an initialization vector for policy parameters that can be fine-tuned when facing new tasks. This system, however, typically utilizes samples of observed online data for its training tasks, discarding relevant past data and leading to performance lag and data inefficiencies. At least one other conventional system attempts to continuously improve upon an underlying parameter initialization vector but does so based on a follow-the-leader algorithm that causes performance lag due to its analysis of all past data whether or not it is relevant to future performance.

In addition, some conventional systems seek to address inaccuracy concerns by modeling underlying transition functions, reward functions, or changes within non-stationary environments to predict future performance of various parameters. However, such an approach requires excessive computational resources. Accordingly, conventional systems often cannot scale with respect to increasing number of states and actions within a complex real-world environment. Indeed, as the complexity of an environment or policy parameterization increase, conventional systems become increasingly inefficient and unable to operate. In addition, many conventional systems update modeling and forecast frequently in an attempt to address the foregoing accuracy concerns. However, in many real-world applications, frequent system updates involve significant computational expense, resulting in excessive and inefficient use of computer resources. Indeed, conventional systems that seek to optimize for the immediate future often lead to sub-optimal utilization of memory and processing power.

The foregoing drawbacks, along with additional technical problems and issues, exist with regard to conventional agent-environment modeling systems.

### SUMMARY

One or more embodiments described herein provide benefits and/or solve one or more of the foregoing or other problems in the art with systems, methods, and non-transitory computer-readable media that flexibly generate a target policy parameter that improves the forecasted future performance of a target policy using a policy gradient algorithm, even in circumstances where the underlying environment is non-stationary. In particular, in one or more embodiments, the disclosed systems configure a target policy for future episodes where an agent decides among available actions using a target policy parameter forecasted to facilitate improved (e.g., near optimal) performance during those episodes. In particular, the disclosed systems can determine a future forecast by fitting a curve to counter-factual estimates of policy performance over time and analyzing performance gradients in these estimates with respect to variations in policy to efficiently generate accurate target policy parameters.

To illustrate, in some implementations, the disclosed systems utilize counter-factual reasoning to estimate what the performance of the target policy would have been if implemented during past episodes. Based on those performance estimates, the disclosed systems forecast the future performance of the target policy during one or more future episodes. Moreover, the disclosed systems can determine gradients indicating how the forecast of the future performance and the past counter-factual estimates will change with respect to variations in the target policy parameter. Utilizing this forward forecasting analysis (based on modeled variations in counter-factual historical performance), the disclosed systems can efficiently and accurately search for a policy that will improve performance without expending the computational expense of expressly modeling the underlying transition functions, reward functions, or non-stationary environmental changes.

Additional features and advantages of one or more embodiments of the present disclosure are outlined in the following description.

### BRIEF DESCRIPTION OF THE DRAWINGS

This disclosure will describe one or more embodiments of the invention with additional specificity and detail by referencing the accompanying figures. The following paragraphs briefly describe those figures, in which:

FIG. 1 illustrates an example environment in which a policy parameter generation system can operate in accordance with one or more embodiments;

FIG. 2 illustrates a diagram of the policy parameter generation system generating a target policy parameter for a target policy in accordance with one or more embodiments;

FIG. 3 illustrates a block diagram for generating counter-factual historical performance metrics for a target policy in accordance with one or more embodiments;

FIG. 4A illustrates a graph indicating forecasted performance metrics generated based on performance trends in accordance with one or more embodiments;

FIG. 4B illustrates a block diagram for generating a forecasted performance metric for a target policy in accordance with one or more embodiments;

FIG. 5 illustrates a block diagram for modifying a target policy parameter of a target policy based on a performance gradient of a forecasted performance metric in accordance with one or more embodiments;

FIG. 6 illustrates a graph displaying weight values applied to counter-factual historical performance metrics determined for a target policy in accordance with one or more embodiments;

FIG. 7 illustrates graphs reflecting experimental results regarding the effectiveness of the policy parameter generation system in accordance with one or more embodiments;

FIG. 8 illustrates an example schematic diagram of a policy parameter generation system in accordance with one or more embodiments;

FIG. 9 illustrates a flowchart of a series of acts for generating a target policy parameter for a target policy in accordance with one or more embodiments; and

FIG. 10 illustrates a block diagram of an exemplary computing device in accordance with one or more embodiments.

### DETAILED DESCRIPTION

One or more embodiments described herein include a policy parameter generation system that flexibly and efficiently adapts future policies to improve future performance, even when the underlying environment is non-stationary. To illustrate, in some implementations, the policy parameter generation system uses counter-factual reasoning to estimate what the performance of the target policy would have been if implemented during past episodes of action-selection. Additionally, the policy parameter generation system fits a regression curve to the counter-factual estimates, modeling the performance trend of the target policy and enabling the forecast of future performance. The policy parameter generation system further differentiates the forecasted future performance to determine how the forecasted future performance changes with respect to changes in the parameter(s) of the target policy. Thus, the policy parameter generation system determines the parameter (or parameter value) that facilitates optimal future performance of the target policy and implements that parameter with the target policy.

To provide an illustration, in one or more embodiments, the policy parameter generation system determines historical performance metrics of a first set of policies applied to a set of previous decision episodes. Utilizing the historical performance metrics, the policy parameter generation system determines a plurality of counter-factual historical performance metrics reflecting application of a target policy having a target policy parameter to the set of previous decision episodes. Further, the policy parameter generation system generates a forecasted performance metric for one or more future decision episodes utilizing the plurality of counter-factual historical performance metrics. The policy parameter generation system also determines a performance gradient of the forecasted performance metric (and historical performance metrics) with respect to varying the target policy parameter. Utilizing the performance gradient of the forecasted performance metric, the policy parameter generation system modifies the target policy parameter of the target policy.

As just mentioned, in one or more embodiments, the policy parameter generation system uses historical performance metrics of a first set of policies to determine counter-factual historical performance metrics for a target policy. In particular, in some implementations, the first set of policies correspond to policies that were previously executed by a digital decision model during a set of previous decision episodes. In some instances, the target policy is different than the policies included in the first set of policies or includes a different policy parameter. In some cases, the policy parameter generation system utilizes counter-factual reasoning to determine what the performance of the target policy would have been during the set of previous decision episodes by determining the counter-factual historical performance metrics for the target policy. Indeed, the policy parameter generation system estimates the performance of the target policy during the set of previous decision episodes though the target policy was not implemented during those previous decision episodes.

In one or more embodiments, the policy parameter generation system utilizes the historical performance metrics of the first set of policies to determine the counter-factual historical performance metrics for the target policy based on reward weights. In particular, in some embodiments, the policy parameter generation system determines reward weights that reflect how actions selected using the first set of policies during the set of previous decision episodes impact the performance of the target policy when used to select those same actions. In one or more embodiments, the policy parameter generation system utilizes an importance sampling estimator to process the historical performance metrics and determine the counter-factual historical performance metrics.

As further mentioned, in some instances, the policy parameter generation system generates a forecasted performance metric for one or more future decision episodes. Indeed, the policy parameter generation system generates the forecasted performance metric to estimate the performance of the target policy for the one or more future decision episodes. As indicated, in some instances, the policy parameter generation system generates the forecasted performance metric utilizing the counter-factual historical performance metrics determined for the target policy. For example, in at least one implementation, the policy parameter generation system generates the forecasted performance metric based on a performance trend of the counter-factual historical performance metrics across the set of previous decision episodes.

In some implementations, the policy parameter generation system utilizes a forecasting model to generate the forecasted performance metric. In some instances, the policy parameter generation system uses a linear forecasting model, such as an identity-based forecasting model, to generate the forecasted performance metric. In some implementations, however, the policy parameter generation system uses a non-linear forecasting model, such as a fourier-based forecasting model.

Additionally, as mentioned above, in one or more embodiments, the policy parameter generation system determines a performance gradient for the forecasted performance metric with respect to varying the target policy parameter. For example, in some implementations, the policy parameter generation system determines changes to the counter-factual historical performance metrics of the target policy with respect to varying the target policy parameter. Further, the policy parameter generation system determines changes to the forecasted performance metric with respect to the changes to the counter-factual historical performance metrics. Thus, in some implementations, the policy parameter generation system determines the performance gradient by combining the changes to the counter-factual historical performance metrics with respect to varying the target policy parameter and the changes to the forecasted performance metric with respect to the changes to the counter-factual historical performance metrics.

In one or more implementations, the policy parameter generation system modifies the target policy parameter of the target policy using the performance gradient determined for the target policy parameter. For example, in some instances, the policy parameter generation system determines a target policy parameter (e.g., a value for the target policy parameter) that improves (e.g., optimizes) the performance of the target policy for the one or more future decision episodes. In some instances, the policy parameter generation system modifies the target policy parameter to improve an average performance metric for the target policy across the one or more future decision episodes. In some implementations, the policy parameter generation system further executes the target policy with the target policy parameter (e.g., the modified target policy parameter) using a digital decision model.

The policy parameter generation system provides several advantages over conventional systems. For example, the policy parameter generation system introduces an unconventional approach to generating target policy parameters that improve the performance of target policies for future decision episodes. To illustrate, the policy parameter generation system utilizes an unconventional ordered combination of actions for estimating how a target policy will perform in future decision episodes based on a performance trend reflected by counter-factual historical performance metrics determined for the target policy. Based on the estimate, the policy parameter generation system determines the target policy that improves that future performance.

Further, the policy parameter generation system operates more flexibly than conventional systems. Indeed, by modifying target policy parameters based on forecasted performances of the respective target policies for future decision episodes, the policy parameter generation system flexibly updates the policies that are implemented to accommodate changes to the environment over time. In particular, in some implementations, the policy parameter generation system flexibly updates the policies before the changes occur.

Additionally, the policy parameter generation system operates more accurately and efficiently than conventional systems. In particular, by updating implemented policies to accommodate changes to the environment (e.g., before the changes even occur), the policy parameter generation system accurately implements policies that promote decisions leading to near optimal rewards. Indeed, the policy parameter generation system avoids the performance lag experienced under many conventional systems. In addition, the policy parameter generation system can utilize a non-uniform weight of data that leverages all available data samples, and thus avoids the data inefficiencies of conventional systems. Further, by determining the target policy parameters that improve forecasted future performance based on a trend of estimated performances for previous decision episodes, the policy parameter generation system accurately determines target policy parameters that are most likely to perform well in future decision episodes.

Further, in some embodiments, the policy parameter generation system further improves efficiency by generating accurate target policy parameters without expressly modeling the underling transition, reward functions, or non-stationary environment changes. Indeed, as outlined in greater detail below, the policy parameter generation system can utilize a univariate time-series to estimate future performance. This approach bypasses the need for modeling the environment and significantly improves efficiency and reduces the burden on computer resources relative to conventional environmental modeling approaches. In addition, by avoiding modeling these underlying functions, the policy parameter generation system can allow for improved scalability in response to a large number of states and actions.

As illustrated by the foregoing discussion, the present disclosure utilizes a variety of terms to describe features and benefits of the policy parameter generation system. Additional detail is now provided regarding the meaning of these terms. For example, as used herein, the term “policy” refers to a set of heuristics, rules, guides, or strategies (e.g., for selection of an action by an agent). In particular, in one or more embodiments, a policy refers to a set of heuristics that guide actions to select when an agent is in particular states. For example, in one or more embodiments, a policy includes a mapping from states to actions or a set of instructions that influence (e.g., dictate) the action selected by an agent from among various actions that are available when the agent is in a particular state. Relatedly, as used herein, the term “target policy” refers to a policy that is under consideration for implementation in the future.

In one or more embodiments, a policy includes at least one policy parameter. As used herein, the term “policy parameter” refers to a particular heuristic, rule, guide, characteristic, feature, or strategy of a policy. In particular, in some instances, a policy parameter refers to a rule that at least partially defines the corresponding policy, such as an action to select when an agent is in a particular state. For example, in some implementations, a policy parameter includes a modifiable or tunable attribute of a policy. Relatedly, as used herein, the term “target policy parameter” refers to a policy parameter of a target policy.

As mentioned above, in one or more embodiments, a policy is associated with an agent, one or more states, and one or more actions. As used herein, the term “agent” refers to a decision maker. In particular, in one or more embodiments, an agent refers to an entity that selects an action (e.g., from among various available actions) when in a particular state. For example, in some cases, an agent includes, but is not limited to, a controller of a mechanical system, a digital system (e.g., that interacts with another digital system or interactions with a person), or a person. As used herein, the term “state” refers to an environmental condition or context. In particular, in one or more embodiments, a state refers to the circumstances of an environment corresponding to an agent at a given point in time. To illustrate, for an agent deciding on digital content to distribute to one or more client devices, a state can include current characteristics or features of the client devices, time features (e.g., day of the week or time of day), previous digital content items distributed to the client devices, etc.

Further, as used herein the term “action” refers to an act performed by an agent. In particular, in one or more embodiments, an action refers to a process (e.g., a sequence of acts), a portion (e.g., a step) of a process, or a single act performed by an agent. To illustrate, in some implementations an action includes sending digital content across a computer network to a computing device.

Further, in one or more embodiments, a policy is associated with a reward. As used herein, the term “reward” (or “policy reward”) refers to a result of an action. In particular, in one or more embodiments, a reward refers to a benefit or response received by an agent for executing a particular action (e.g., while in a particular state). In some implementations, a reward refers to a benefit or response received for executing a sequence or combination of actions. For example, in some instances, a reward includes a response to a recommendation (e.g., following the recommendation), an interaction with distributed digital content (e.g., viewing the digital content or clicking on a link provided within the digital content), progress towards a goal, or an improvement to some metric.

In some implementations, the policy parameter generation system executes a policy as part of a Markov Decision Process. Accordingly, as used herein, the term “Markov Decision Process reward” refers to a reward associated with a Markov Decision Process that is obtained in response to a selection of one or more actions. Relatedly, as used herein, the term “forecasted Markov Decision Process reward” refers to a reward that is predicted to be obtained in response to a selection of one or more actions in association with a Markov Decision Process.

In one or more embodiments, the policy parameter generation system utilizes a digital decision model to execute a policy. As used herein, the term “digital decision model” refers to a computer-implemented model or algorithm that executes policies. In particular, in one or more embodiments, a digital decision model includes a computer-implemented model that selects a particular action when in a particular state in accordance with a policy. In some instances, a digital decision model is the agent that executes the actions and moves from state to state in response. In some embodiments, a digital decision model interacts with the agent to provide recommendations of actions for the agent to select.

As used herein, the term “decision episode” refers to a decision event. In particular, in one or more embodiments, a decision episode refers to an instance in which an agent has an opportunity to select an action. For example, in some implementations, a decision episode includes an occurrence of an agent selecting an action from among multiple available actions while in a given state or an occurrence of the agent selecting the only action available while in a given state. As used herein, the term “previous decision episode” refers to a past decision episode in which a policy was executed. By contrast, as used herein, the term “future decision episode” refers to a decision episode that will occur in the future. For example, in some implementations, a future decision episode refers to a decision episode in which a target episode will potentially be executed in the future. Relatedly, as used herein, the term “time duration” refers to an interval that includes one or more decision episodes. For example, in some implementations, a time duration refers to a period of time (e.g., a day, a week, a month, etc.) that spans one or more decision episodes. In some instances, a time duration refers to a pre-determined number of decision episodes.

Additionally, as used herein, the term “performance metric” refers to a standard for measuring, evaluating, or otherwise reflecting the performance of a policy. In particular, in one or more embodiments, a performance metric includes a value that corresponds to some attribution of policy performance. For example, in some instances, a performance metric refers to a reward resulting from selection of an action in accordance with a policy or a cumulative award resulting from the combination of actions selected in accordance with the policy. A performance metric is often associated with additional information regarding a state, event, and/or action. For example, a performance metric can reflect a reward resulting from one or more states associated with a policy (e.g., the set of states associated with the agent during execution of the policy), one or more of the actions associated with the policy (e.g., the set of actions selected by the agent during execution of the policy), or one or more probabilities for selecting the actions (e.g., the probabilities of selecting each available action while in a particular state). As used herein, the term “historical performance metric” refers to a performance metric associated with a policy that have previously been executed. In contrast, as used herein, the term “forecasted performance metric” refers to a performance metric predicted to be associated with a policy (e.g., a target policy) during execution in the future. As used herein, the term “average performance metric” refers to a value that reflects the average or mean of a corresponding performance metric throughout the execution of a policy. For example, in some instances, an average performance metric includes an average reward received during a time duration for execution of a policy.

Relatedly, as used herein, the term “counter-factual historical performance metric” refers to an estimated performance metric for a policy applied to previous decision episodes in which a different policy was actually executed. In particular, in one or more embodiments, a counter-factual historical performance metric refers to a performance metric that reflects application to a target policy to one or more previous decision episodes to which the target policy was not actually applied. Indeed, in one or more embodiments, the policy parameter generation system utilizes counter-factual reasoning to estimate (e.g., via a counter-factual historical performance metric) how a target policy would have performed if the target policy had been applied to a previous decision episode.

Additionally, as used herein, the term “importance sampling estimator” refers to a computer-implemented model or algorithm estimates how a policy would have performed had that policy been implemented during a particular decision episode. In particular, in one or more embodiments, an importance sampling estimator refers to a computer-implemented algorithm that implements counter-factual reasoning to estimate the performance of a policy during a decision episode using the performance of another policy during the decision episode. For example, in some implementations, an importance sampling estimator includes a computer-implemented algorithm that determines a counter-factual historical performance metric reflecting application of a target policy during a decision episode based on a historical performance metric of another policy that was actually applied during the decision episode. In some implementations, an importance sampling estimator includes a per-decision importance sampling estimator (“PDIS”). In some cases, an importance sampling estimator includes a weighted importance sampling estimator.

Further, as used herein the term “reward weight” refers to a value reflecting the comparative impact of a particular reward or performance metric (and/or an action or policy corresponding to the performance metric). In particular, in one or more embodiments, a reward weight refers to a value reflecting a performance impact of an action selection in accordance with one policy compared to a performance impact of the action selection in accordance with another policy. For example, in one or more implementations, a reward weight includes a value reflecting a comparison between a performance impact of an action selected using a target policy while in a state and a performance impact of the action selected using a different policy while in the state.

As used herein, the term “performance trend” refers to a trend associated with a plurality of performance metrics across one or more decision episodes. In particular, in one or more embodiments, a performance trend refers to a pattern of performance of a policy applied to one or more decision episodes (e.g., a sequence of decision episodes). For example, a performance trend can include a line or curve fitted to a set of data samples. For example, in some instances, a performance trend reflects a best-fit curve fit to historical performance metrics (e.g., measured historical performance metrics of a policy and/or counter-factual historical performance metrics of a policy).

Additionally, as used herein, the term “performance gradient” refers to a value that represents a change in the performance with respect to changes in a policy/policy parameter. In particular, in one or more embodiments, a performance gradient refers to a value that represents a change in the performance metric of a policy in response to a change in one or more other attributes or characteristics of policy execution. For example, in some implementations, a performance gradient includes a value that reflects a change in the forecasted performance metric of a target policy with respect to variations in the target policy parameter of the target policy. Similarly, a performance gradient can include a value that reflects a change in counter-factual historical performance metrics with respect to variations in the target policy parameter of the target policy.

As used herein, the term “forecasting model” refers to a computer-implemented model or algorithm that determines forecasted performance metrics. In particular, in one or more embodiments, a forecasting model refers to a computer-implemented model that determines forecasted performance metrics for a target policy conditioned on (e.g., using) counter-factual historical performance metrics associated with the target policy. For example, in some instances, a forecasting model includes an ordinary least squares (“OLS”) regression model, a simple linear regression model, a multiple linear regression model, a straight line model, or a moving average model. In some implementations, a forecasting model includes a linear model, such as an identity-based forecasting model. In some instances, however, a forecasting model includes a non-linear model, such as a fourier-based forecasting model.

Additionally, as used herein, the term “entropy regularizer value” refers to a metric or parameter that represents one or more unknown or unexpected elements. In particular, in one or more embodiments, an entropy regularizer value refers to a parameter included in an algorithm that reflects a degree of randomness in the real world. For example, in some implementations, the policy parameter generation system utilizes an entropy regularizer value when updating a target policy parameter of a target policy to improve the performance of the target policy during one or more future decision episodes despite the introduction of one or more unknown or unexpected elements during the decision episode(s). Relatedly, as used herein, the term “noise component” refers to the one or more unknown or unexpected elements.

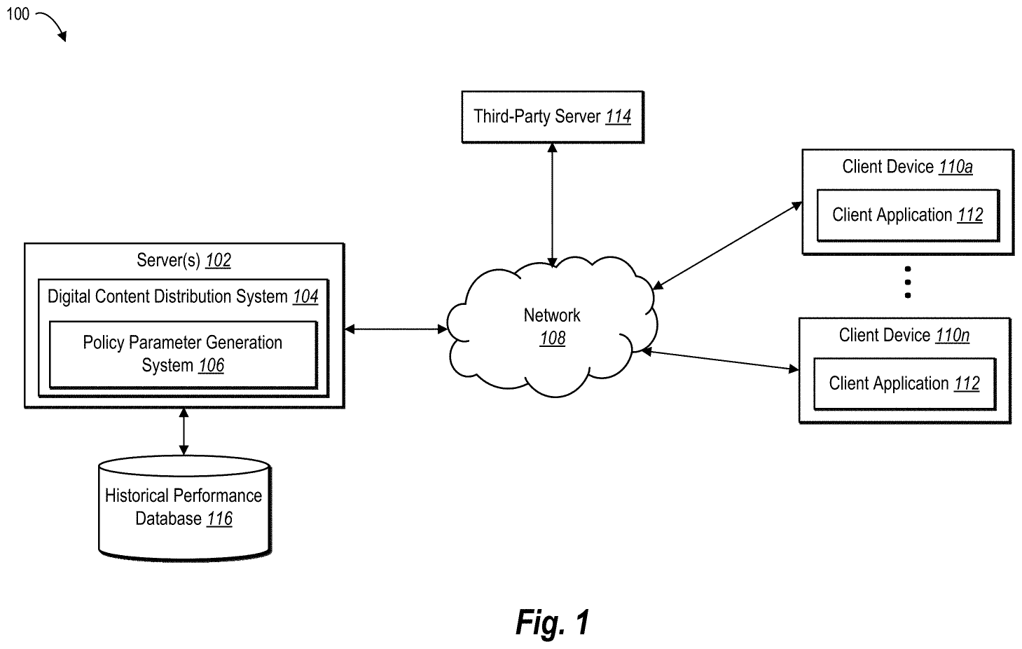

Additional detail regarding the policy parameter generation system will now be provided with reference to the figures. For example, FIG. 1 illustrates a schematic diagram of an exemplary system environment (“environment”) 100 in which a policy parameter generation system 106 can be implemented. As illustrated in FIG. 1, the environment 100 includes a server(s) 102, a network 108, client devices 110a-110n, a third-party server 114, and a historical performance database 116.

Although the environment 100 of FIG. 1 is depicted as having a particular number of components, the environment 100 can have any number of additional or alternative components (e.g., any number of servers, client devices, third-party servers, historical performance databases, or other components in communication with the policy parameter generation system 106 via the network 108). Similarly, although FIG. 1 illustrates a particular arrangement of the server(s) 102, the network 108, the client devices 110a-110n, the third-party server 114, and the historical performance database 116, various additional arrangements are possible.

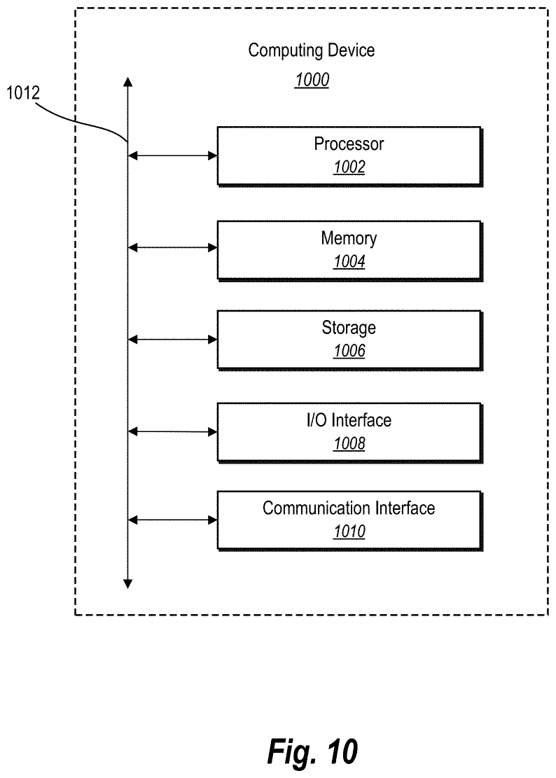

The server(s) 102, the network, 108, the client devices 110a-110n, the third-party server 114, and the historical performance database 116 may be communicatively coupled with each other either directly or indirectly (e.g., through the network 108 discussed in greater detail below in relation to FIG. 10). Moreover, the server(s) 102, the client devices 110a-110n, and the third-party server 114 may include a variety of computing devices (including one or more computing devices as discussed in greater detail with relation to FIG. 10).

As mentioned above, the environment 100 includes the server(s) 102. In one or more embodiments, the server(s) 102 generate, store, receive, and/or transmit digital data, including digital data related to the implementation of policies. To provide an illustration, in some instances, the sever transmits, to a client device (e.g., one of the client devices 110a-110n), digital data related to an action selected in accordance with a policy, such as a recommendation or digital content selected to be distributed to the client device. In some implementations, the server transmits digital data related to a selected action to a third-party system (e.g., hosted on the third-party server 114). In one or more embodiments, the server(s) 102 comprises a data server. In some embodiments, the server(s) 102 comprises a communication server or a web-hosting server.

As shown in FIG. 1, the server(s) 102 includes the digital content distribution system 104. In one or more embodiments, the digital content distribution system 104 provides functionality for distributing digital content to a third-party system or a user via a client device. To illustrate, in some implementations, the digital content distribution system 104 identifies or otherwise determines information associated with a client device (e.g., the client device geographic location, user characteristics corresponding to the client device, etc.). Accordingly, the digital content distribution system 104 distributes, to a client device associated, digital content that tailored to the specific characteristics or features of the client devices and in accordance with a particular digital policy.

Additionally, the server(s) 102 includes the policy parameter generation system 106. In particular, in one or more embodiments, the policy parameter generation system 106 utilizes the server(s) 102 to generate target policy parameters for target policies. For example, in some instances, the policy parameter generation system 106 utilizes the server(s) 102 to determine historical performance metrics for a first set of previously-applied policies and use the historical performance metrics to generate a target policy parameter for a target policy.

To illustrate, in one or more embodiments, the policy parameter generation system 106, via the server(s) 102, determines historical performance metrics of a first set of policies applied to (e.g., executed during) a set of previous decision episodes. The policy parameter generation system 106, via the server(s) 102, further utilizes the historical performance metrics to determine plurality of counter-factual historical performance metrics reflecting application of a target policy having a target policy parameter to the set of previous decision episodes. Via the server(s) 102, the policy parameter generation system 106 also generates a forecasted performance metric for one or more future decision episodes utilizing the plurality of counter-factual historical performance metrics and determines a performance gradient of the forecasted performance metric with respect to varying the target policy parameter. Further, the policy parameter generation system 106, via the server(s) 102, modifies the target policy parameter of the target policy utilizing the performance gradient of the forecasted performance metric.

In one or more embodiments, the third-party server 114 interacts with the policy parameter generation system 106, via the server(s) 102, over the network 108. For example, in some instances, the third-party server 114 hosts a third-party system and receives recommendations for actions for the third-party system to take from the policy parameter generation system 106 in accordance with a policy implemented by the policy parameter generation system 106. In some instances, the third-party server 114 receives, from the policy parameter generation system 106, instructions for optimizing the parameters of the third-party server 114 in accordance with an implemented policy. In some instances, the third-party server 114 receives digital data, such as digital content, in response to the policy parameter generation system 106 selecting a particular action.

In one or more embodiments, the historical performance database 116 stores historical performance metrics of policies applied to previous decision episodes. As an example, in some instances, the historical performance database 116 stores historical performance metrics provided by the policy parameter generation system 106 after executing policies. The historical performance database 116 further provides access to the historical performance metrics to the policy parameter generation system 106. Though FIG. 1 illustrates the historical performance database 116 as a distinct component, one or more embodiments include the historical performance database 116 as a component of the server(s) 102, the digital content distribution system 104, or the policy parameter generation system 106.

In one or more embodiments, the client devices 110a-110n include computing devices that are capable of receiving digital data related to actions selected in accordance with a policy (e.g., recommendations for actions to take, distributed digital content, etc.). For example, in some implementations, the client devices 110a-110n include at least one of a smartphone, a tablet, a desktop computer, a laptop computer, a head-mounted-display device, or other electronic devices. In some instances, the client devices 110a-110n include one or more applications (e.g., the client applications 112) that are capable of receiving digital data related to actions selected in accordance with a policy. For example, in some embodiments, the client application 112 includes a software application installed on the client devices 110a-110n. In other cases, however, the client application 112 includes a web browser or other application that accesses a software application hosted on the server(s) 102.

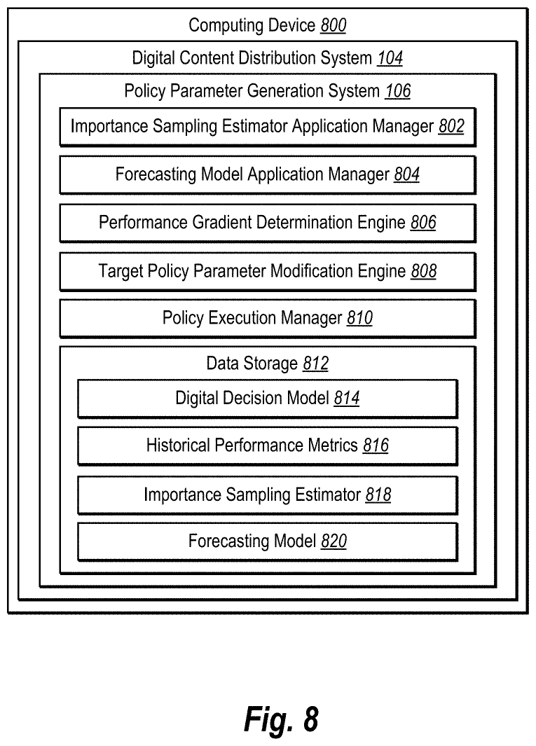

The policy parameter generation system 106 can be implemented in whole, or in part, by the individual elements of the environment 100. Indeed, although FIG. 1 illustrates the policy parameter generation system 106 implemented with regard to the server(s) 102, different components of the policy parameter generation system 106 can be implemented by a variety of devices within the environment 100. For example, one or more (or all) components of the policy parameter generation system 106 can be implemented by a different computing device (e.g., one of the client devices 110a-110n) or a separate server from the server(s) 102 hosting the digital content distribution system 104 (e.g., the third-party server 114). Example components of the policy parameter generation system 106 will be described below with regard to FIG. 8.

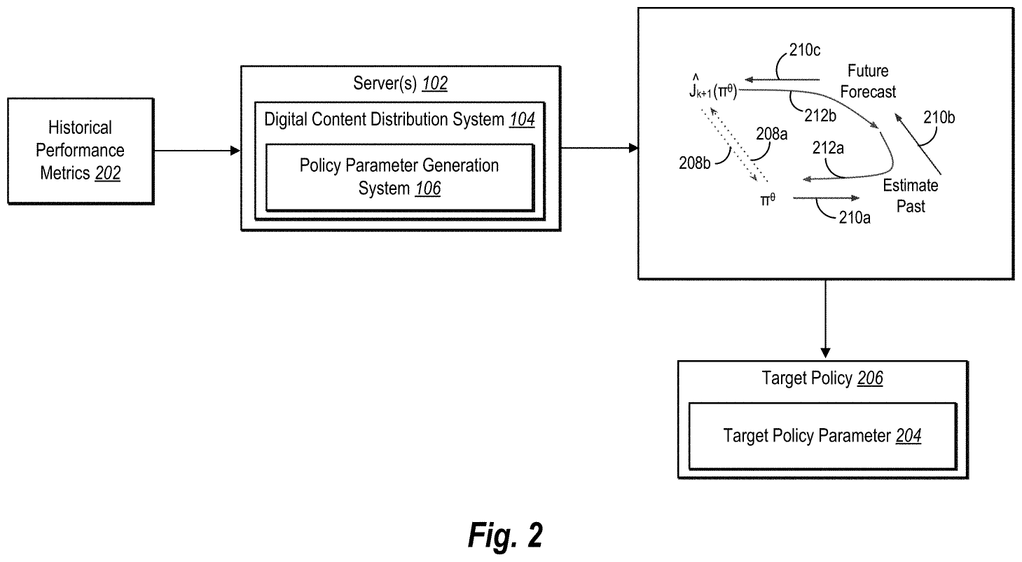

As mentioned above, the policy parameter generation system 106 generates (e.g., modifies) target policy parameters for target policies to be applied to future decision episodes. FIG. 2 illustrates an overview diagram of the policy parameter generation system 106 generating a target policy parameter for a target policy in accordance with one or more embodiments.

As shown in FIG. 2, the policy parameter generation system 106 determines (e.g., identifies) historical performance metrics 202. In particular, in one or more embodiments, the historical performance metrics 202 correspond to the historical performance metrics of a set of policies applied to a set of previous decision episodes. For example, in some implementations, the historical performance metrics 202 correspond to the historical performance metrics of one or more policies applied to a set of the most recently executed decision episodes. Indeed, in some implementations, the historical performance metrics 202 include historical performance metrics associated with a several different policies applied to previous decision episodes. For example, in some implementations, a first subset of the historical performance metrics 202 can correspond to a first policy applied to previous decision episodes, and a second subset of the historical performance metrics 202 can correspond to a second policy applied to previous decision episodes.

In one or more embodiments, the policy parameter generation system 106 determines (e.g., identifies) the historical performance metrics 202 by accessing a database storing the historical performance metrics 202. For example, in some implementations, the policy parameter generation system 106 maintains a database that stores historical performance metrics for subsequent access. In some instances, the policy parameter generation system 106 receives or retrieves the historical performance metrics 202 from another platform (e.g., a third-party system) that executes policies and tracks the corresponding performance metrics.

As further shown in FIG. 2, the policy parameter generation system 106 generates a target policy parameter 204 of a target policy 206. In particular, as shown by the dashed arrow 208a of FIG. 2, the policy parameter generation system 106 determines a forecasted performance (represented as Ĵₖ₊₁(π^{θ})) of the target policy 206 (represented as π^{θ}) for one or more future decision episodes. Accordingly, the policy parameter generation system 106 generates the target policy parameter 204 (represented as θ) based on the forecasted performance. In one or more embodiments, as data for the future decision episode(s) is unavailable, the policy parameter generation system 106 determines the forecasted performance of the target policy 206 utilizing the historical performance metrics 202. Indeed, because the data for the future decision episode(s) cannot be obtained, the policy parameter generation system 106 does not determine the forecasted performance of the target policy 206 directly (as is indicated by the dashed arrow 208a). Rather, in such embodiments, the policy parameter generation system 106 utilizes an indirect approach of determining the forecasted performance using the historical performance metrics 202 (as indicated by the arrows 210a-210c).

For example, as illustrated by the arrow 210a of FIG. 2, the policy parameter generation system 106 determines the forecasted performance of the target policy 206 by estimating a past performance of the target policy. In particular, in some cases, the policy parameter generation system 106 utilizes the historical performance metrics 202 to estimate the performance of the target policy 206 had the target policy 206 been applied to the set of previous decision episodes corresponding to the historical performance metrics 202. For example, in one or more embodiments, the policy parameter generation system 106 processes the historical performance metrics 202 to generate a plurality of counter-factual historical metrics reflecting application of the target policy 206 to the set of previous decision episodes.

Further, as illustrated by the arrow 210b of FIG. 2, the policy parameter generation system 106 determines a forecast for the future based on the estimate of the past performance. In particular, in one or more embodiments, the policy parameter generation system 106 determines the forecast for the future based on the counter-factual historical performance metrics determined for the target policy 206. For example, in some instances, the counter-factual historical performance metrics indicate a performance trend across the previous decision episodes and provides a forecast indicating how the performance trend continues into future decision episodes.

Additionally, as shown by the arrow 210c of FIG. 2, based on the forecast for the future, the policy parameter generation system 106 determines the forecasted performance of the target policy 206 for the one or more decision episodes. For example, in some implementations, the policy parameter generation system 106 determines the forecasted performance of the target policy 206 using the counter-factual historical performance metrics determined for the target policy 206 (e.g., based on the performance trend indicated by the counter-factual historical performance metrics). In some instances, the policy parameter generation system 106 determines the forecasted performance of the target policy 206 by generating a forecasted performance metric for the target policy 206. Though FIG. 2 illustrates determining the forecast for the future and generating the forecasted performance of the target policy 206 as separate acts, it should be understood that the policy parameter generation system 106 performs these acts together in some embodiments. In other words, by generating the forecasted performance of the target policy 206, the policy parameter generation system 106 determines the forecast for the future.

As shown by the dashed arrow 208b of FIG. 2, the policy parameter generation system 106 generates the target policy parameter 204 of the target policy 206 by further returning to the target policy 206 for additional analysis. Indeed, in one or more embodiments, the policy parameter generation system 106 determines variability or changes to the forecasted performance metric of the target policy 206 based on change or variability of the target policy parameter 204. However, in one or more embodiments, the policy parameter generation system 106 does not determine the changes to the forecasted performance metric directly (as suggested by the dashed arrow 208b). Rather, in such embodiments, the policy parameter generation system 106 utilizes performance gradients (as suggested by the arrows 212a-212b).

In particular, in one or more embodiments, the policy parameter generation system 106 further analyzes the target policy 206 to determine a policy gradient for the future performance metric generated for the target policy 206. In other words, the policy parameter generation system 106 determines how the forecasted performance metric for the target policy 206 changes with respect to changes to the target policy parameter 204 (e.g., changes to the value of the target policy parameter 204). For example, as shown by the arrow 212a of FIG. 2, the policy parameter generation system 106 varies the target policy parameter 204 (e.g., varies the value of the target policy parameter 204) and determines the changes to the counter-factual historical performance metrics of the target policy 206. Further, as shown by the arrow 212b of FIG. 2, the policy parameter generation system 106 determines the changes to the future performance metric of the target policy 206 based on the changes to the counter-factual historical performance metrics. In some cases, based on the performance gradient of the forecasted performance metric, the policy parameter generation system 106 generates the target policy parameter 204 (e.g., modifies the value of the target policy parameter 204).

Accordingly, in some implementations, the policy parameter generation system 106 determines the target policy parameter 204 that improves the forecasted performance of the target policy 206 for the one or more future decision episodes. To illustrate, in one or more embodiments, the target policy 206 includes a particular value (e.g., a default value or previously-implemented value) for the target policy parameter 204. The policy parameter generation system 106 determines another value of the target policy parameter 204 that improves the forecasted performance of the target policy 206 for the one or more future decision episodes using the performance gradient. Accordingly, the policy parameter generation system 106 modifies the target policy parameter 204 to include the other value.

Though not shown in FIG. 2, in one or more embodiments, the policy parameter generation system 106 executes the target policy 206 with the target policy parameter 204 for the one or more decision episodes. For example, in some implementations, the policy parameter generation system 106 utilizes a digital decision model to execute the target policy 206.

As mentioned above, in some implementations, the policy parameter generation system 106 executes policies (e.g., the target policy 206 or the set of policies applied to the previous decision episodes) as part of a Markov Decision Process (“MDP”). In some instances, the policy parameter generation system 106 represents an MDP as a tuple (S, A, P, R, γ, d⁰) where S represents the set of possible states, A represents the possible actions, P represents a transition function, R represents a reward function, γ represents a discount factor, and d⁰ represents a start state distribution. The policy parameter generation system 106 utilizes R(s, a) to represent an expected reward resulting from selecting to execute action a while in state s. For a given set X, the policy parameter generation system 106 utilizes Δ(X) to represent the set of distributions over X. For example, in one or more embodiments, the policy parameter generation system 106 treats a policy π: S→Δ(A) as the distribution of actions conditioned on the state.

As suggested above, in some implementations, the policy parameter generation system 106 utilizes π^{θ} (as will be used in the discussion below) to indicate that the target policy π is parameterized using θϵ [symbol] ^{d}. Further, in a non-stationary setting, as the MDP changes over time, the policy parameter generation system 106 utilizes Mₖ to denote the MDP used in decision episode k. Further, the policy parameter generation system 106 utilizes the super-script t to represent the time-step within an episode. Accordingly, Sₖ^{t}, Aₖ^{t}, and Rₖ^{t} represent random variables corresponding to the state, the action, and the reward, respectively, at time step t in episode k. Further, Hₖ represents a trajectory in episode k: (sₖ⁰, aₖ⁰, rₖ⁰, sₖ¹, aₖ¹, . . . , sₖ^{T}), where T is the finite horizon.

In one or more embodiments, the policy parameter generation system 106 also uses vₖ^{π}^{^{θ}}(s)= [symbol] [Σⱼ₌₀^{T-t}γ^{j}Rₖ^{t+j}|Sₖ^{t}=s, π^{θ}] as the value function evaluated at state s, during episode k, under the policy π, where conditioning on π denotes that the trajectory in episode k is sampled using π. Further, in some instances, the policy parameter generation system 106 uses Jₖ (π^{θ}):=Σₛ d₀(s) vₖ^{π}^{^{θ}}(s) for the start state objective for policy π in episode k. Accordingly, in some cases, the policy parameter generation system 106 uses J*ₖ=max_{π}Jₖ(π^{θ}) to represent the performance of the optimal policy for Mₖ.

In one or more embodiments, to model non-stationarity where the environment in which a policy is executed changes, the policy parameter generation system 106 allows an exogeneous process change the MDP from Mₖ to Mₖ₊₁ (i.e., between decision episodes). In some instances, the policy parameter generation system 106 utilizes {Mₖ}ₖ₌₁^{∞} to represent a sequence of MPDs where each MDP Mₖ is denoted by the tuple (S, A, Pₖ, Rₖ, γ, d⁰). As suggested by the tuple, in some implementations, the policy parameter generation system 106 determines that, for any two MDPs Mₖ and Mₖ₊₁, the state set S, the action set A, the starting distribution d⁰ and the discount factor γ are the same. Further, in some cases, the policy parameter generation system 106 determines that both the transition dynamics (P₁, P₂, . . . ) and the reward functions (R₁, R₂, . . . ) vary smoothly over time.

In accordance with the above, in one or more embodiments, the policy parameter generation system 106 identifies or otherwise determines target policies that improve the regret obtained from executing policies across decision episodes. In particular, in some embodiments, the policy parameter generation system 106 identifies or otherwise determines target policy parameters of target policies that improve the regret. As such, in some implementations, the policy parameter generation system 106 generally operates to determine a sequence of target policies (e.g., of target policy parameters) that minimizes the lifelong regret of executing those target policies as follows:

$${argmin}_{\{ {π}_{1}^{θ} , … {π}_{k}^{θ} , … \}} {\sum }_{k = 1}^{∞} {J}_{k}^{*} - {\sum }_{k = 1}^{∞} {J}_{k} ( {π}_{k}^{θ} ) \qquad ( 1 )$$

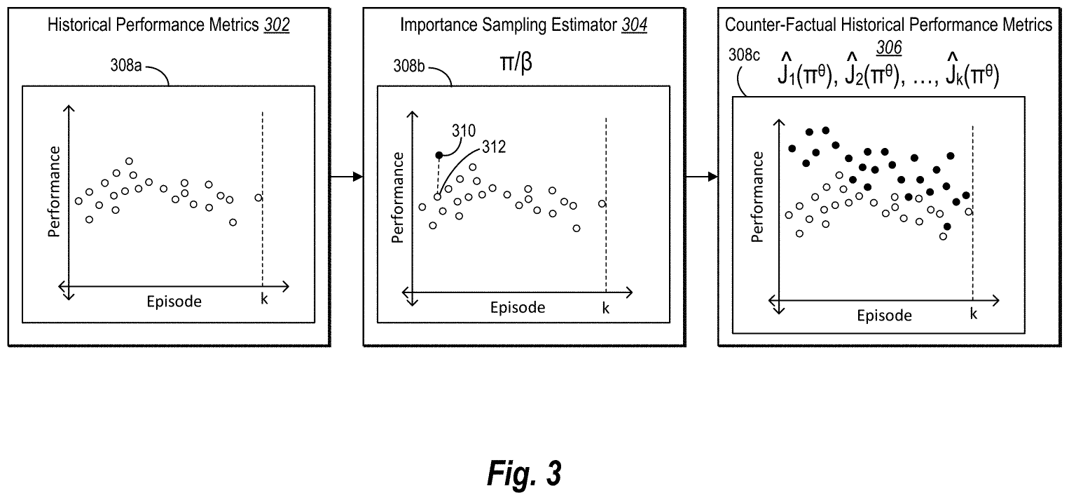

As mentioned above, in one or more embodiments, the policy parameter generation system 106 estimates a past performance for a target policy. For example, in some instances, the policy parameter generation system 106 generates counter-factual historical performance metrics reflecting application of the target policy to previous decision episodes. FIG. 3 illustrates a block diagram for generating counter-factual historical performance metrics for a target policy in accordance with one or more embodiments.

As shown in FIG. 3, the policy parameter generation system 106 determines the historical performance metrics 302 of a set of policies applied to a set of previous decision episodes (as discussed above with reference to FIG. 2). In particular, as illustrated by the graph 308a, the historical performance metrics 302 reflect the performance of the policy executed during each previous decision episode from the set of previous decision episodes. As further shown by the graph 308a, in some implementations, the performance of a policy executed during a given previous decision episode can differ from the performance of a policy executed during a different previous decision episode. In some instances, the performance of a policy executed during a given previous decision episode differs from the performance of the same policy executed during a different decision episode. Indeed, as previously mentioned, in some instances, changes to the environment in which a policy is executed affect the performance of the policy.

Further, as shown in FIG. 3, the policy parameter generation system 106 processes the historical performance metrics 302 of the set of policies using an importance sampling estimator 304. In particular, the policy parameter generation system 106 utilizes the importance sampling estimator 304 to generate counter-factual historical performance metrics 306 for a target policy having a target policy parameter (e.g., a default value or previously-implemented value) based on the historical performance metrics 302. In one or more embodiments, the counter-factual historical performance metrics 306 reflect application of the target policy to the set of previous decision episodes to which the historical performance metrics 302 correspond.

Indeed, as discussed above, in one or more embodiments, the policy parameter generation system 106 determines that the transition dynamics (P₁, P₂, . . . ) and the reward functions (R₁, R₂, . . . ) associated with policies implemented within an environment vary smoothly over time. Accordingly, in some instances, the policy parameter generation system 106 further determines that the performances (J₁(π^{θ}), J₂(π^{θ}), . . . ) of a given policy will also vary smoothly over time. In other words, the policy parameter generation system 106 determines that smooth changes in the environment result in smooth changes to the performance of a policy. Accordingly, the policy parameter generation system 106 aims to analyze the performance trend of a policy over previous decision episodes to identify a policy (e.g., identify a policy parameter for the policy) that provides desirable performance for future decision episodes.

In some implementations, however, the target policy includes a new policy that was not applied to the set of previous decision episodes. Therefore, in some cases, the policy parameter generation system 106 does not determine the true values of the past performances J_{1:k}(π^{θ}) for the target policy; rather, the policy parameter generation system 106 determines estimated past performances Ĵ_{1:k}(π^{θ}). In other words, the policy parameter generation system 106 determines an estimate of how the target policy would have performed if the target policy were applied to the set of previous decision episodes. In one or more embodiments, the policy parameter generation system 106 determines this estimate by utilizing the importance sampling estimator 304 to generate the counter-factual historical performance metrics 306 for the target policy using the historical performance metrics 302.

Indeed, in one or more embodiments, for a non-stationary MDP starting with a fixed transition matrix P₁ and a reward function R₁, the policy parameter generation system 106 determines that the performance Jᵢ(π^{θ}) of a target policy π for a decision episode i≤k is generally represented as follows where P₁ and R₁ are random variables:

$${J}_{i} ( {π}^{θ} ) = {\sum }_{t = 0}^{T} {γ}^{t} 𝔼 [ {R}_{i}^{t} \vert {π}^{θ} , {P}_{1} , {R}_{1} ] \qquad ( 2 )$$

In one or more embodiments, to obtain the estimate Ĵᵢ(π^{θ}) of the target policy π's performance during episode i, the policy parameter generation system 106 utilizes the past trajectory Hᵢ of the i^{th} episode that was observed when executing policy βᵢ. Accordingly, in some implementations, the policy parameter generation system 106 determines (e.g., using the important sampling estimator 304) the estimate Ĵᵢ(π^{θ}) as follows:

$${\hat{J}}_{i} ( {π}^{θ} ) := {\sum }_{t = 0}^{H} ( {\prod }_{i = 0}^{t} \frac{{π}^{θ} ( {A}_{i}^{l} \vert {S}_{i}^{l} )}{{β}_{i} ( {A}_{i}^{l} \vert {S}_{i}^{l} )} ) {γ}^{t} {R}_{i}^{t} \qquad ( 3 )$$

In equation 3, π^{θ}(Aᵢ^{l}|Sᵢ^{l})/βᵢ(Aᵢ^{l}|Sᵢ^{l}) represents a reward weight that reflects a comparison between a first performance impact of an action selected using the target policy π^{θ} while in a state and a second performance impact of the action selected using the policy βᵢ while in the state. As mentioned above, in one or more embodiments, the reward weight corresponds to a weight applied to the reward Rᵢ^{t}, to indicate the importance (e.g., the performance impact) of actions selected using the policy βᵢ compared to the importance of those actions under the target policy π^{θ}. In other words, the policy parameter generation system 106 utilizes the reward weight implemented by the importance sampling estimator 304 to indicate at least one attribute of a relationship between the target policy π^{θ} and the policy βᵢ. In particular, as illustrated by the graph 308b, the policy parameter generation system 106 utilizes a relationship between the performances of the target policy π^{θ} and the policy βᵢ as shown by the relationship between the performance indicator 310 for the target policy π^{θ} and the performance indicator 312 for the policy βᵢ.

As suggested by equation 3 and as illustrated in FIG. 3, by processing the historical performance metrics 302, the policy parameter generation system 106 does not process the set of policies (e.g., the policy βᵢ) applied to the set of previous decision episodes themselves. Indeed, as indicated in equation 3, the policy parameter generation system 106 uses the states associated with the policies, the actions associated with the policies, and the rewards resulting from those actions. In some implementations, the policy parameter generation system 106 further uses the probabilities for selecting the actions under the policies.

Thus, as shown in FIG. 3, the policy parameter generation system 106 generates the counter-factual historical performance metrics 306 for the target policy. Indeed, as illustrated by the graph 308c, in some instances, the policy parameter generation system 106 generates a counter-factual historical performance metric corresponding to each historical performance metric from the historical performance metrics 302.

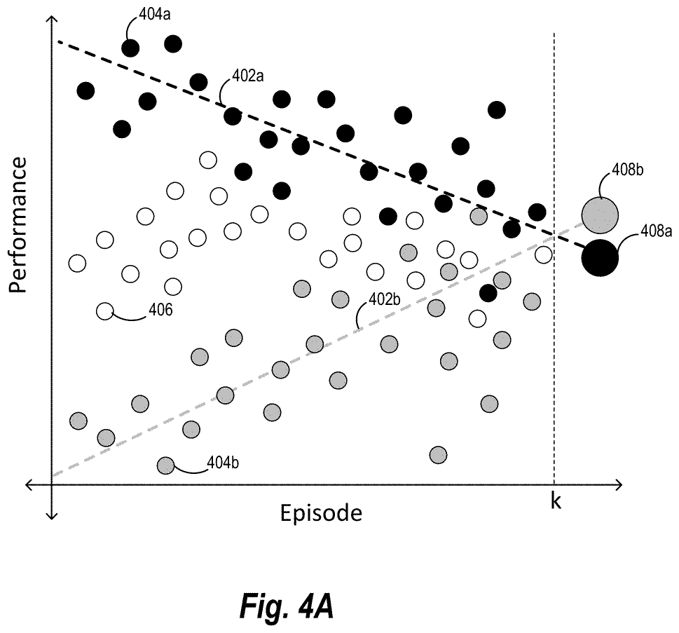

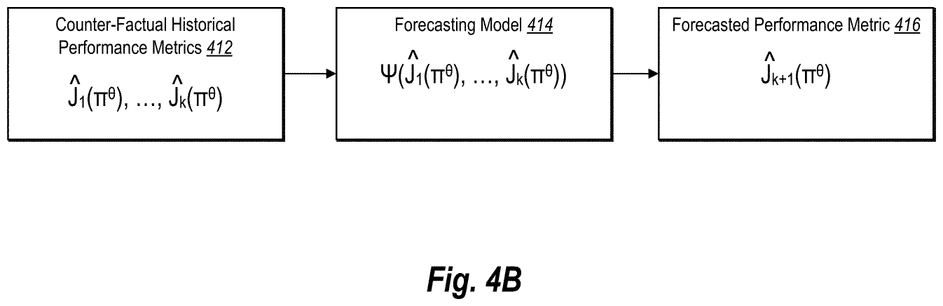

As previously discussed, in one or more embodiments, the policy parameter generation system 106 generates a forecasted performance metric for the target policy utilizing the counter-factual historical performance metrics determined for the target policy. For example, in some implementations, the policy parameter generation system 106 generates the forecasted performance metric based on a performance trend indicated by the counter-factual historical performance metrics. FIGS. 4A-4B illustrate diagrams for generating a forecasted performance metric for a target policy in accordance with one or more embodiments.

In particular, FIG. 4A illustrates a graph indicating forecasted performance metrics generated based on performance trends in accordance with one or more embodiments. For example, the graph of FIG. 4A illustrates a performance trend 402a indicated by a first set of counter-factual historical performance metrics (e.g., the set including the counter-factual historical performance metric 404a). In one or more embodiments, the policy parameter generation system 106 determines the first set of counter-factual historical performance metrics using historical performance metrics (e.g., the set including the historical performance metric 406) of a set of policies applied to a set of previous decision episodes as discussed above with reference to FIG. 3. In some instances, based on the performance trend 402a, the policy parameter generation system 106 generates the forecasted performance metric 408a for the corresponding target policy.

Further, the graph of FIG. 4A illustrates a performance trend 402b indicated by a second set of counter-factual historical performance metrics (e.g., the set including the counter-factual historical performance metric 404b). In some embodiments, the policy parameter generation system 106 determines the second set of counter-factual historical performance metrics using the historical performance metrics of the set of policies applied to the set of previous decision episodes as discussed above with reference to FIG. 3. In some instances, based on the performance trend 402b, the policy parameter generation system 106 generates the forecasted performance metric 408b for the corresponding target policy. Indeed, as indicated by the graph of FIG. 4A, in one or more embodiments, the policy parameter generation system 106 generates forecasted performance metrics for multiple target policies (or a target policy having different default or previously-implemented values).

As further indicated by the graph of FIG. 4A, in some implementations, a first target policy having higher counter-factual historical performance metrics compared to a second target policy may have a lower forecasted performance metric than the second target policy. For example, the performance trend of the first target policy may indicate decreasing performance across time that results in a lower forecasted performance metric while the performance trend of the second target policy may indicate improving performance that results in a higher forecasted performance metric. Thus, by generating a forecasted performance metric for a target policy based on a performance trend of the counter-factual historical performance metrics determined for that target policy, the policy parameter generation system 106 ensures implementation of a target policy that will provide good performance for future decision episodes despite potentially poor estimated past performance. In particular, the policy parameter generation system 106 accurately determines target policy parameters that are likely to perform well (e.g., provide near optimal performance) during the future decision episodes.

FIG. 4B illustrates a block diagram for generating a forecasted performance metric for a target policy in accordance with one or more embodiments. In particular, as shown in FIG. 4B, the policy parameter generation system 106 utilizes a forecasting model 414 to process counter-factual historical performance metrics 412 determined for a target policy and generate a forecasted performance metric 416 for the target policy.

For exampling, in one or more embodiments, the policy parameter generation system 106 utilizes the forecasting model 414 to generate the forecasted performance metric for the target policy as follows:

Ĵₖ₊₁(π^{θ}):=Ψ(Ĵ₁(π^{θ}),Ĵ₂(π^{θ}), . . . ,Ĵₖ(π^{θ})) (4)

In equation 4, Ψ( ) represents the forecasting model 414. As discussed above, the forecasting model 414 can include one of various available forecasting models. For example, in at least one implementation, the forecasting model 414 includes an OLS regression model having parameters wϵ [symbol] ^{d×1}. In one or more embodiments, the policy parameter generation system 106 provides the forecasting model 414 with the following inputs:

X:=[1,2, . . . ,k]^{T}ϵ [symbol] ^{k×1} (5)

Y:=[Ĵ₁(π^{θ}),Ĵ₂(π^{θ}),Ĵ₃(π^{θ}), . . . ,Ĵₖ(π^{θ})]^{T}ϵ [symbol] ^{k×1} (6)

In one or more embodiments, for any xϵX, the policy parameter generation system 106 utilizes ϕ(x)ϵ [symbol] ^{1×d} to denote a d-dimensional basis function for encoding the time index. In some instances, the policy parameter generation system 106 utilizes one of the following as the basis function:

ϕ(x):={x,1} (7)

ϕ(x):={sin(2π^{θ}nx|nϵ [symbol] _{>0})}∪{cos(2π^{θ}nx|nϵ [symbol] _{>0})}∪{1} (8)

In particular, equation 7 indicates an identity basis function, and equation 8 represents a fourier basis function. Accordingly, in one or more embodiments, the policy parameter generation system 106 utilizes, as the forecasting model 414, an identity-based forecasting model (e.g., by implementing equation 7). Further, in some embodiments, the policy parameter generation system 106 utilizes, as the forecasting model 414, a fourier-based forecasting model (e.g., by implementing equation 8). However, it should be noted that the policy parameter generation system 106 can implement various other linear or non-linear forecasting models in other embodiments.

In some implementations, the policy parameter generation system 106 utilizes Φϵ [symbol] ^{k×d} as the basis matrix corresponding to the implemented basis function. Accordingly, the policy parameter generation system 106 uses w=(Φ^{T}Φ)⁻¹Φ^{T}Y as the solution to the least squares regression problem provided by equation 4. Accordingly, in one or more embodiments, the policy parameter generation system 106 generates the forecasted performance metric as follows:

Ĵₖ₊₁(π^{θ})=ϕ(k+1)w=ϕ(k+1)(Φ^{T}Φ)⁻¹Φ^{T}Y (9)

In one or more embodiments, by using a univariate time series to generate the forecasted performance metric, the policy parameter generation system 106 estimates the future performance of a target policy without modeling the environment itself. Thus, the policy parameter generation system 106 operates more flexibly than conventional systems that require modeling of the environment, including the underlying transition or reward functions. Further, it should be noted that Φ^{T}Φϵ [symbol] ^{d×d} where d<<k, in some cases, making the cost of computing the inverse matrix negligible. Accordingly, the policy parameter generation system 106 provides improved flexibility and efficiency over conventional systems as the policy parameter generation system 106 can scale to more challenging problems while being robust to the size of the state set S or the action set A.

Though FIG. 4B illustrates the policy parameter generation system 106 generating the forecasted performance metric 416 based on the counter-factual historical performance metrics 412 alone, it should be noted that the policy parameter generation system 106 can generate the forecasted performance metric 416 utilizing additional metrics in some instances. For example, in some embodiments, the policy parameter generation system 106 applies the target policy to one or more previous decision episodes. In particular, the historical performance metrics from which the counter-factual historical performance metrics 412 were generated can include one or more historical performance metrics associated with the target policy itself. In such embodiments, the policy parameter generation system 106 utilizes these historical performance metrics of the target policy with the counter-factual historical performance metrics 412 to generate the forecasted performance metric 416 for the target policy.

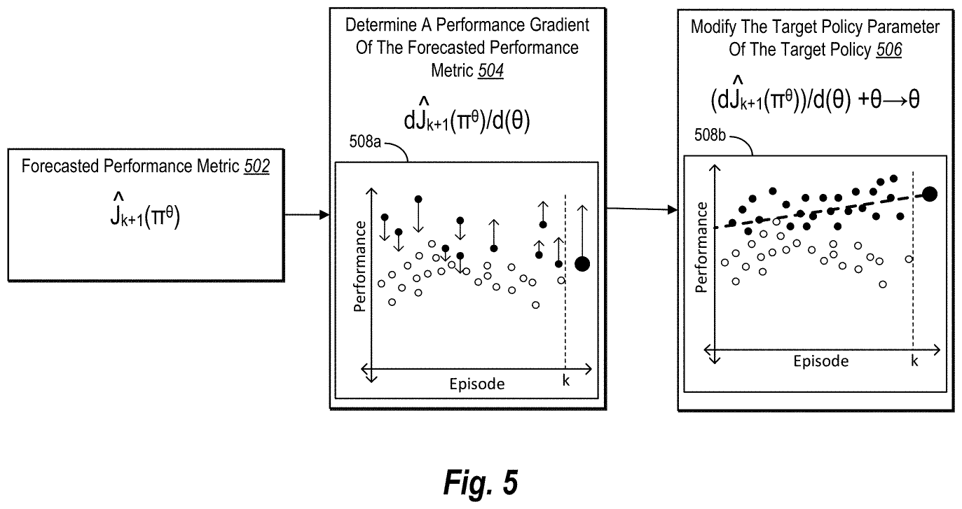

As discussed above, in some implementations, the policy parameter generation system 106 utilizes the forecasted performance metric for a target policy to modify a target policy parameter of the target policy. In particular, the policy parameter generation system 106 determines a performance gradient of the forecasted performance metric and modifies the target policy parameter based on the performance gradient. FIG. 5 illustrates a block diagram for modifying a target policy parameter of a target policy based on a performance gradient of a forecasted performance metric in accordance with one or more embodiments.

For example, as shown in FIG. 5, the policy parameter generation system 106 determines a forecasted performance metric 502 for a target policy as discussed above with reference to FIGS. 4A-4B. Further, as shown in FIG. 5, the policy parameter generation system 106 performs an act 504 of determining a performance gradient of the forecasted performance metric. In one or more embodiments, the policy parameter generation system 106 determines the performance gradient of a forecasted performance metric as follows:

$$\frac{d {\hat{J}}_{k = 1} ( {π}^{θ} )}{d θ} = \frac{d Ψ ( {\hat{J}}_{1} ( {π}^{θ} ) , … , {\hat{J}}_{k} ( {π}^{θ} ) )}{d θ} \qquad ( 10 )$$

In some implementations, the policy parameter generation system 106 expands equation 10 as follows:

$$\frac{d {\hat{J}}_{k = 1} ( {π}^{θ} )}{d θ} = {\sum }_{i = 1}^{k} \frac{d Ψ ( {\hat{J}}_{1} ( {π}^{θ} ) , … , {\hat{J}}_{k} ( {π}^{θ} ) )}{\partial {\hat{J}}_{i} ( {π}^{θ} )} \cdot \frac{d {\hat{J}}_{i} ( {π}^{θ} )}{d θ} \qquad ( 11 )$$

The first term in equation 11 represents changes to the estimated future performance of the target policy with respect to changes in the estimated past performance of the target policy. In particular, the first term represents changes to the forecasted performance metric of the target policy with respect to changes to the past outcomes (e.g., the counter-factual historical performance metrics determined for the target policy). Further, the second term in equation 11 represents changes to the estimated past performance of the target policy with respect to changes in the target policy parameter of the target policy. In particular, the second term represents changes to the counter-factual historical performance metrics determined for the target policy with respect to varying the target policy parameter. As indicated by equation 11, in some implementations, the policy parameter generation system combines the changes to the plurality of counter-factual historical performance metrics and the changes to the forecasted performance metric to determine the performance gradient.

In other words, in one or more embodiments, the policy parameter generation system 106 varies the value of the target policy parameter (e.g., by taking a derivative with respect to the policy parameter). Further, as indicated by the graph 508a, the policy parameter generation system 106 determines how the counter-factual historical performance metrics and the forecasted performance metric change in response to the variations. Accordingly, the policy parameter generation system 106 determines the performance gradient based on these changes.

In one or more embodiments, in order to obtain the first term of equation 11, the policy parameter generation system 106 leverages equation 4 and the correspondence between Ĵᵢ(π^{θ}) and the i^{th} element of Y as follows where [Z]ᵢ represents the i^{th} element of a vector Z:

$$\frac{d {\hat{J}}_{k + 1} ( {π}^{θ} )}{\partial {\hat{J}}_{i} ( {π}^{θ} )} = \frac{\partial ϕ ( k + 1 ) {( {Φ}^{⊤} Φ )}^{- 1} {Φ}^{⊤} Y}{\partial {Y}_{i}} = {[ ϕ ( k + 1 ) {( {Φ}^{⊤} Φ )}^{- 1} {Φ}^{⊤} Y ]}_{i} \qquad ( 12 )$$

To obtain the second term of equation 11, in one or more embodiments, the policy parameter generation system 106 determines that ρᵢ(0,l):=Πⱼ₌₀^{l}π^{θ}(Aᵢ^{l}|Sᵢ^{l})/βᵢ(Aᵢ^{l}|Sᵢ^{l}). Accordingly, in some cases, the policy parameter generation system 106 obtains the second term of equation 11 as follows:

$$\frac{d {\hat{J}}_{i} ( {π}^{θ} )}{d θ} = {\sum }_{t = 0}^{T} \frac{\partial {logπ}^{θ} ( {A}_{i}^{t} \vert {S}_{i}^{t} )}{\partial θ} ( {\sum }_{l = t}^{T} {ρ}_{i} ( 0 , l ) {γ}^{l} {R}_{i}^{l} ) \qquad ( 13 )$$

As further shown in FIG. 5, the policy parameter generation system 106 performs an act 506 of modifying the target policy parameter of the target policy. For example, in some implementations, the policy parameter generation system 106 modifies the target policy parameter based on the performance gradient determined for the forecasted performance metric. Indeed, in some implementations, by determining the performance gradient, the policy parameter generation system 106 determines a value for the target policy parameter having a performance trend that indicates that the target policy will provide improved performance for one or more future decision episodes (e.g., as illustrated by the graph 508b). In some embodiments, the policy parameter generation system 106 modifies the target policy parameter to include the value that corresponds to the highest forecasted performance metric for the target policy. In at least one implementation, the policy parameter generation system 106 modifies the target policy parameter to improve an average performance metric for the target policy across the one or more future decision episodes for which the target policy will be implemented.

In some implementations, the policy parameter generation system 106 utilizes the modified target policy to reprocess the historical performance metrics of the set of policies applied to the set of previous decision episodes. For example, in some implementations, the policy parameter generation system 106 utilizes the historical performance metrics to determine an additional plurality of counter-factual historical performance metrics reflecting application of the modified target policy to the set of previous decision episodes, generate an additional forecasted performance metric for the one or more future decision episodes utilizing the additional plurality of counter-factual historical performance metrics, and change the modified target policy parameter utilizing an additional performance gradient of the additional forecasted performance metric. Indeed, in some embodiments, the policy parameter generation system 106 iteratively determines a performance gradient for a forecasted performance metric and modifies the target policy parameter accordingly to further improve the forecasted performance of the target policy.

In some implementations, the policy parameter generation system 106 determines a time duration for executing a given policy. For example, in some instances, the policy parameter generation system 106 determines a time duration that spans one or more decision episodes and corresponds to an interval used for executing a given policy before modifying the policy or implementing a new policy. Accordingly, when implemented, the policy parameter generation system 106 executes the target policy within the time duration. In some implementations, the policy parameter generation system 106 modifies the target policy parameter to improve an average performance metric for the target policy within the time duration. In one or more implementations, the policy parameter generation system 106 utilizes a tunable hyperparameter to determines the time duration. Accordingly, the policy parameter generation system 106 operates flexibly in that the policy parameter generation system 106 can modify the length into the future for which it optimizes the performance (e.g., improves the average performance metric) of the target policy. In some implementations, where δ represents the determined time duration, the policy parameter generation system 106 minimizes the lifelong regret provided by equation 1 by modifying the target policy parameter to improve the average performance metric of the target policy as follows:

$${argmax}_{{π}^{θ}} ( 1 / δ ) {\sum }_{Δ = 1}^{δ} {\hat{J}}_{k + Δ} ( {π}^{θ} ) \qquad ( 14 )$$

In some embodiments, the policy parameter generation system 106 further modifies the target policy parameter using an entropy regularizer value. In particular, in some implementations, the policy parameter generation system 106 utilizes an entropy regularizer value to avoid having the target policy become too deterministic, precluding the agent from exploring states that were previously undesirable but may have become more rewarding due to the changes in the environment. Further, in some cases, by utilizing the entropy regularizer value, the policy parameter generation system 106 mitigates the high variances potentially generated by the importance sampling estimator when the target policy is too deterministic. Thus, in one or more embodiments, the entropy regularizer value corresponds to a noise component that prevents the target policy from becoming too deterministic. Accordingly, in some implementations, the policy parameter generation system 106 further determines an entropy regularizer value (represented as H) and modifies the target policy parameter of the target policy based on the performance gradient of the forecasted performance metric and the entropy regularizer value.

The algorithm presented below is another description of how the policy parameter generation system 106 generates generate (e.g., modifies) a target policy parameter for a target policy in some embodiments.

```
Algorithm 1
Input Learning-rate η, time-duration δ, entropy-regularizer λ
Initialize Forecasting function Ψ, Buffer [symbol]
while True do
  #Record a new batch of trajectories using π^{θ}
  for episode 1, 2, . . . , δ do
    h = {(s_{0:T}, a_{0:T}, Pr(s_{0:T}|a_{0:T}), r_{0:T})} [symbol]
    .insert (h)
  #Update for future performance
  for i = 1, 2, . . . do
    #Evaluate past performances
    for k = 1, 2, . . . , | [symbol]   | do
      Ĵₖ(π^{θ}) = Σₜ₌₀^{T} ρᵢ(0, t)γ^{t}Rₖ^{t}
    #Future forecast and its gradient [symbol]
    (π^{θ}) = 1/δΣ_{Δ=1}^{δ} Ĵ_{k+Δ}(π^{θ})
    $θ ← θ + η \frac{\partial }{\partial θ} ( ℒ ( {π}^{θ} ) + λ H ( {π}^{θ} ) )$
```

By generating (e.g., modifying) a target policy parameter based on a forecasted performance metric for a target policy, the policy parameter generation system 106 operates more flexibly than conventional systems. Indeed, by forecasting the performance of a target policy for future decision episodes and modifying the target policy parameter using the forecast, the policy parameter generation system 106 flexibly accommodates changes to an environment. Further, generating a target policy parameter based on forecasted performance enables improved accuracy over conventional systems. For example, because the policy parameter generation system 106 generates the target policy parameter in the manner described above, the policy parameter generation system 106 avoids the performance lag experienced by many conventional systems.

Thus, in one or more embodiments, the policy parameter generation system 106 determines a target policy parameter for a target policy. In particular, the policy parameter generation system 106 determines the target policy parameter based on an estimate of the performance of the target policy during one or more future decision episodes. Further, the policy parameter generation system 106 generates the estimate for the target policy based on historical performance metrics of other policies applied to previous decision episodes. Accordingly, in some implementations, the algorithm and acts described with reference to FIGS. 2-5 comprise the corresponding structure for performing a step for determining a target policy parameter for a target policy for one or more future decision episodes to be executed by the digital decision model from the historical performance metrics of the first set of policies for the set of previous decision episodes.

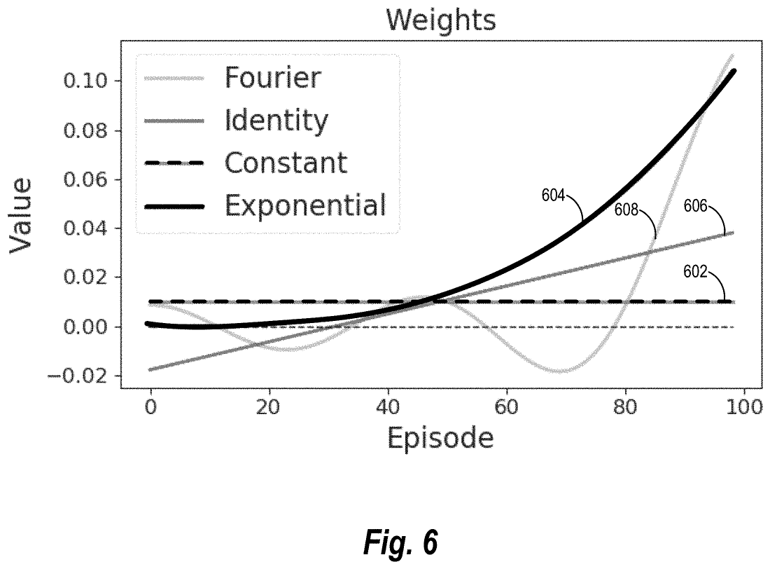

As discussed above, in some instances, the policy parameter generation system 106 generates a forecasted performance metric for a target policy based on a performance trend indicated by counter-factual historical performance metrics determined for the target policy. In one or more embodiments, the policy parameter generation system 106 applies weights to the counter-factual historical performance metrics determined for a target policy and generates the forecasted performance metric based on the weighted counter-factual historical performance metrics. FIG. 6 illustrates a graph displaying weight values applied to counter-factual historical performance metrics determined for a target policy in accordance with one or more embodiments.

For example, in one or more embodiments, in determining the performance gradient of a forecasted performance metric, the policy parameter generation system 106 multiplies the first term in equation 11 (e.g., the gradient of future performance) by the second term of equation 11 (e.g., the gradient provided by the importance sampling estimator—such as a PDIS gradient term). Accordingly, in some embodiments, the policy parameter generation system 106 treats the performance gradient of the forecasted performance metric as a weighted sum of off-policy policy gradients. FIG. 6 illustrates a graph of the weights ∂Ĵ₁₀₀(π^{θ})/∂Ĵᵢ(π^{θ}) for importance sampling estimator gradients of each episode i, when the performance for the one hundredth decision episode is forecasted using data from the past ninety-nine decision episodes. In one or more embodiments, where the importance sampling estimator includes an OLS regression model, the weights are independent of Y from equation 6.

The graph of FIG. 6 provides a qualitative comparison of weights provided by various embodiments of the policy parameter generation system 106 and weights provided by one or more conventional systems. For example, the curve 602 represents the weights provided by one or more conventional systems that implement an existing online-based algorithm, such as the follow-the-leader algorithm. As illustrated by the curve 602, these systems maximize performance on all of the past data uniformly. Additionally, the curve 604 represents the weights provided by conventional systems implementing an exponential approach. In particular, these systems typically only optimize performance using data from recent episodes and largely discard previous data. As suggested by the graph of FIG. 6, the approaches corresponding to the curves 602, 604 only use non-negative weights. Accordingly, implementing systems may fail to properly capture the trend associated with a target policy. For example, these implementing systems may fail to determine that a first target policy with worse past performance than a second target policy is likely to provide better future performance than the second target policy.

In contrast, the curve 606 corresponds to at least one embodiment of the policy parameter generation system 106 utilizing an identity-based forecasting model to generate a forecasted performance metric for a target policy. As illustrated by the curve 606, in some implementations, the policy parameter generation system 106 utilizes the identity-based forecasting model to minimize performances in the distance past and maximize performances in the recent past. Accordingly, by using an identity-based forecasting model, the policy parameter generation system 106 can identify those target policies whose performance is on a linear rise, expecting those target policies to provide improved performance in future decision episodes.

Additionally, the curve 608 corresponds to at least one embodiment of the policy parameter generation system 106 utilizing a fourier-based forecasting model. As illustrated by the curve 608, in some implementations, the policy parameter generation system 106 utilizes the fourier-based forecasting model to apply weights with alternative positive/negative signs. Accordingly, by using the fourier-based forecasting model, the policy parameter generation system 106 takes into account the sequential differences in performances over the past, thereby favoring the target policy that shows the most performance increments in the past. Further, by using the fourier-based forecasting model, the policy parameter generation system 106 avoids restricting the performance trend of a target policy to be linear.

Though the above discusses the policy parameter generation system 106 operating in a non-stationary environment, the policy parameter generation system 106 can operate in stationary environments in some embodiments. For example, in one or more embodiments, if J(π) represents the performance of a policy for a stationary MDP, Ĵ_{k+δ}(π) represents the non-stationary importance sampling estimators of performance 6 decision episodes in the future, ϕ represents the basis function used to encode the time index in the forecasting model Ψ, then the policy parameter generation system 106 satisfies the following two conditions: ϕ(⋅) contains 1 to incorporate a bias/intercept coefficient in least-squares regression (e.g., ϕ(⋅)=[ϕ₁(⋅), . . . , ϕ_{d-1}(⋅), 1], where ϕ(⋅) are arbitrary functions); and Φ has full column ranks such that (Φ^{T}Φ)⁻¹ exists. Accordingly, in one or more embodiments, the policy parameter generation system 106 includes the following attribute: for all δ≥1, J_{k+δ}(π) is an unbiased estimator of J(π), that is [symbol] [Ĵ_{k+δ}(π)]=J(π). In some embodiments, the policy parameter generation system 106 further includes the following attribute: for all δ≥1, Ĵ_{k+δ}(π) is a consistent estimator of J(π), that is

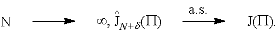

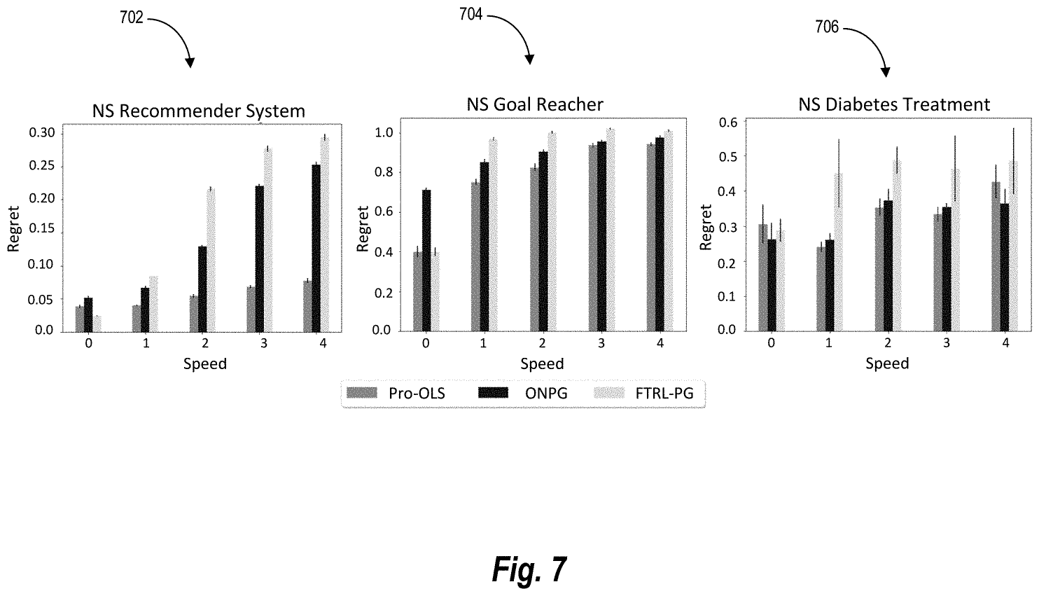

As mentioned above, in one or more embodiments, the policy parameter generation system 106 operates more accurately than conventional systems. In particular, by updating implemented policies to accommodate changes to the environment, the policy parameter generation system accurately implements policies that promote decisions leading to near optimal rewards. Researchers have conducted studies to determine the accuracy of at least one embodiment of the policy parameter generation system 106. FIG. 7 illustrates graphs reflecting experimental results regarding the effectiveness of the policy parameter generation system 106 in accordance with one or more embodiments.

Specifically, the graphs of FIG. 7 compare the performance of one embodiment of the policy parameter generation system 106 (labeled “Pro-OLS”) to the performance of one model (labeled “ONPG”) that performs purely online optimization by fine-tuning the existing policy using only the trajectory being observed online. The graphs further include the performance of another model (labeled “FTRL-PG”) that implements follow-the-regularized-leader optimization by maximizing performance over both the current and all the past trajectories.

The graphs of FIG. 7 illustrate the performance of each tested model in three different environments inspired by real-world applications that exhibit non-stationarity. For example, the graph 702 corresponds to a non-stationary recommender system in which a recommender engine interacts with a user whose interest in different items fluctuates over time. Further, the rewards associated with each item vary in seasonal cycles. The goal of the models in this environment is to maximize the revenue obtained by recommending an item that the user is most interested in at any given time.

The graph 704 corresponds to a non-stationary goal reacher consisting of a two-dimensional environment with four (e.g., down, up, left, right) available actions and a continuous state representing the Cartesian coordinates. The goal of the tested models in this environment is to make the agent reach a moving goal post.

The graph 706 corresponds to a non-stationary environment in which diabetes treatment is administered. In particular, the environment is based on an open-source implementation of the FDA approved Type-1 Diabetes Mellitus simulator (“T1DMS”) for treatment of type-1 diabetes. Each decision episode corresponds to a day in an in-silico patient's life. Consumption of a meal increases the blood-glucose level in the body. The patient can suffer from hyperglycemia or hypoglycemia depending on whether the patient's blood-glucose level becomes too high or too low, respectively. The goal of the tested models is to control the blood-glucose level of the patient by regulating the insulin dosage to minimize the risk of hyperglycemia and hypoglycemia. It should be noted that, in such an environment, the insulin sensitivity of a patient's internal body organs varies over time, inducing the non-stationarity. In the T1DMS simulator, the researchers induced this non-stationarity by oscillating the body parameters (e.g., insulin sensitivity, rate of glucose absorption, etc.) between two known configurations available in the simulator.

In each of the environments, the researchers further regulated the speed of non-stationarity to test each model's ability to adapt. A higher speed corresponds to a greater amount of non-stationarity. A speed of zero indicates that the environment is stationary.

In the non-stationary recommender system, as the exact value of J*ₖ is available from the simulator, the researchers could determine the true value of regret. For the non-stationary goal reacher and the non-stationary diabetes treatment environments, however, J*ₖ is not known for any k, so the researchers used a surrogate measure for regret. Accordingly, \tilde{J}*ₖ represents the maximum return obtained in episode k by any algorithm and (Σₖ₌₁^{N}(\tilde{J}*ₖ−Jₖ(π)))/(Σₖ₌₁^{N}\tilde{J}*ₖ) represents the surrogate regret for a policy π.

As shown by the graphs 702, 704, 706, the policy parameter generation system 106 generally performs better (i.e., with less regret) than the other tested models. In particular, even when all tested models provide comparative performance when the environment is stationary (i.e., the speed is set to 0), the performance of the ONPG and FTRL-PG models typically deteriorates worse than the policy parameter generation system 106 as the speed of non-stationarity increases. Indeed, the policy parameter generation system 106 leverages the past data to better capture the non-stationarity, and thus more robustly accommodates changes to the environments. Notably, the FTRL-PG experiences a significant amount of performance lag due to its consideration of all past data equally.

As the foregoing examples discussed with reference to FIG. 7 suggest, the policy parameter generation system 106 can operate within a variety of environments and can implement policies (e.g., target policies) promoting a variety of corresponding actions. For example, in some implementations, an action includes administering a medication (or a particular dose of a medication) to a patient. In some implementations, an action includes movement in a particular direction. In some cases, an action includes providing a recommendation of a particular product or service to a client device.

Turning now to FIG. 8, additional detail will now be provided regarding various components and capabilities of the policy parameter generation system 106. In particular, FIG. 8 illustrates the policy parameter generation system 106 implemented by the computing device 800 (e.g., the server(s) 102 and/or one of the client devices 110a-110n discussed above with reference to FIG. 1). Additionally, the policy parameter generation system 106 is also part of the digital content distribution system 104. As shown, in one or more embodiments, the policy parameter generation system 106 includes, but is not limited to an importance sampling estimator application manager 802, a forecasting model application manager 804, a performance gradient determination engine 806, a target policy parameter modification engine 808, a policy execution manager 810, and data storage 812 (which includes a digital decision model 814, historical performance metrics 816, an importance sampling estimator 818, and a forecasting model 820).

As just mentioned, and as illustrated by FIG. 8, the policy parameter generation system 106 includes the importance sampling estimator application manager 802. In particular, the importance sampling estimator application manager 802 determines counter-factual historical performance metrics reflecting application of a target policy applied to a set of previous decision models. For example, in one or more embodiments, the importance sampling estimator application manager 802 utilizes an importance sampling estimator to generate the counter-factual historical performance metrics based on historical performance metrics of a first set of policies applied to the set of previous decision episodes.

Additionally, as shown in FIG. 8, the policy parameter generation system 106 includes the forecasting model application manager 804. In particular, the forecasting model application manager 804 generates a forecasted performance metric for a target policy. For example, in one or more embodiments, the forecasting model application manager 804 generates the forecasted performance metric using the counter-factual historical performance metrics determined for the target policy by the importance sampling estimator application manager 802. To illustrate, in some implementations, the forecasting model application manager 804 generates the forecasted performance metric based on a performance trend indicated by the counter-factual historical performance metrics.

As shown in FIG. 8, the policy parameter generation system 106 further includes the performance gradient determination engine 806. In particular, the performance gradient determination engine 806 determines a performance gradient for a forecasted performance metric generated for a target policy by the forecasting model application manager 804. For example, in some implementations, the performance gradient determination engine 806 varies the target policy parameter of the target policy and determines the resulting changes to counter-factual historical performance metrics of the target policy. The performance gradient determination engine 806 further determines the changes to the forecasted performance metric for the target policy based on the changes to the counter-factual historical performance metrics. The performance gradient determination engine 806 combines the changes to determine the performance gradient.

Further, as shown in FIG. 8, the policy parameter generation system 106 includes the target policy parameter modification engine 808. In particular, the target policy parameter modification engine 808 modifies the target policy parameter of a target policy based on the performance gradient of the forecasted performance metric determined for the target policy by the performance gradient determination engine 806. In one or more embodiments, the target policy parameter modification engine 808 modifies the target policy parameter to improve the forecasted performance metric for the target policy across one or more future decision episodes. In some implementations, the target policy parameter modification engine 808 modifies the target policy parameter to improve an average performance metric of the target policy within a time duration that spans one or more future decision episodes.

As shown in FIG. 8, the policy parameter generation system 106 also includes the policy execution manager 810. In particular, the policy execution manager 810 executes policies within corresponding environment. For example, in some implementations, the policy execution manager 810 executes a target policy having a modified target policy parameter across one or more decision episodes. In some instances, the policy execution manager 810 utilizes a digital decision model to execute policies.

As further shown in FIG. 8, the policy parameter generation system 106 includes data storage 812. In particular, data storage 812 includes the digital decision model 814, the historical performance metrics 816, the importance sampling estimator 818, and the forecasting model 820. In one or more embodiments, the digital decision model 814 stores the digital decision model utilizes by the policy execution manager 810 to execute policies. In some embodiments, the historical performance metrics 816 includes the historical performance metrics of policies applied to previous decision episodes. In one or more implementations, the importance sampling estimator 818 stores the importance sampling estimator utilizes by the importance sampling estimator application manager 802 to generate counter-factual historical performance metrics for a target policy (e.g., based on historical performance metrics stored by the historical performance metrics 816). In one or more embodiments, the forecasting model 820 stores the forecasting model utilized by the forecasting model application manager 804 to generate a forecasted performance metric for a target policy.

Each of the components 802-820 of the policy parameter generation system 106 can include software, hardware, or both. For example, the components 802-820 can include one or more instructions stored on a computer-readable storage medium and executable by processors of one or more computing devices, such as a client device or server device. When executed by the one or more processors, the computer-executable instructions of the policy parameter generation system 106 can cause the computing device(s) to perform the methods described herein. Alternatively, the components 802-820 can include hardware, such as a special-purpose processing device to perform a certain function or group of functions. Alternatively, the components 802-820 of the policy parameter generation system 106 can include a combination of computer-executable instructions and hardware.

Furthermore, the components 802-820 of the policy parameter generation system 106 may, for example, be implemented as one or more operating systems, as one or more stand-alone applications, as one or more modules of an application, as one or more plug-ins, as one or more library functions or functions that may be called by other applications, and/or as a cloud-computing model. Thus, the components 802-820 of the policy parameter generation system 106 may be implemented as a stand-alone application, such as a desktop or mobile application. Furthermore, the components 802-820 of the policy parameter generation system 106 may be implemented as one or more web-based applications hosted on a remote server. Alternatively, or additionally, the components 802-820 of the policy parameter generation system 106 may be implemented in a suite of mobile device applications or “apps.” For example, in one or more embodiments, the policy parameter generation system 106 can comprise or operate in connection with digital software applications such as ADOBE® TARGET, ADOBE® ANALYTICS, or ADOBE® SENSEI™. “ADOBE,” “TARGET,” “ANALYTICS,” and “SENSEI” are either registered trademarks or trademarks of Adobe Inc. in the United States and/or other countries.

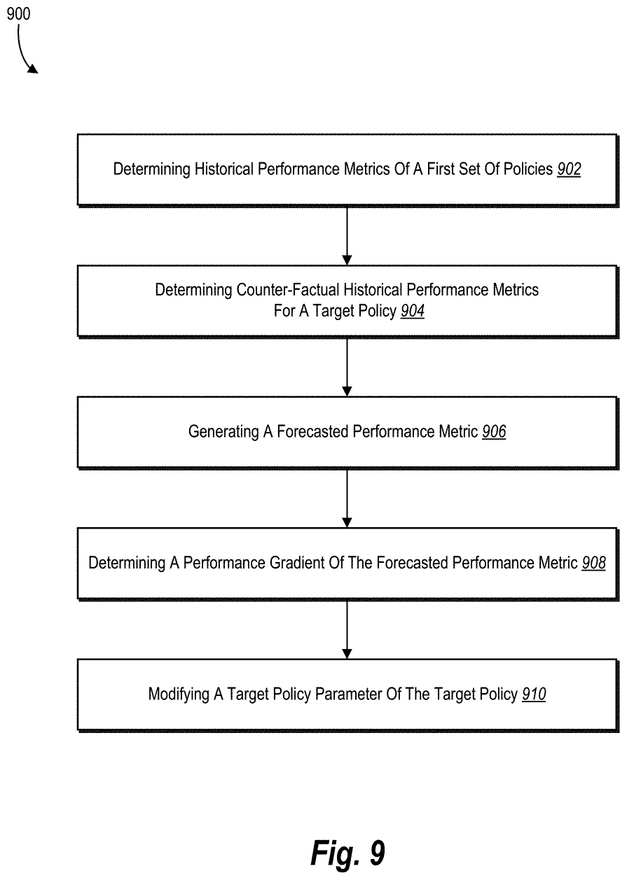

FIGS. 1-8, the corresponding text and the examples provide a number of different methods, systems, devices, and non-transitory computer-readable media of the policy parameter generation system 106. In addition to the foregoing, one or more embodiments can also be described in terms of flowcharts comprising acts for accomplishing particular results, as shown in FIG. 9. FIG. 9 may be performed with more or fewer acts. Further, the acts may be performed in different orders. Additionally, the acts described herein may be repeated or performed in parallel with one another or in parallel with different instances of the same or similar acts.

FIG. 9 illustrates a flowchart of a series of acts 900 for generating (e.g., modifying) a target policy parameter for a target policy in accordance with one or more embodiments. While FIG. 9 illustrates acts according to one embodiment, alternative embodiments may omit, add to, reorder, and/or modify any of the acts shown in FIG. 9. In some implementations, the acts of FIG. 9 are performed as part of a method. For example, in some embodiments, the acts of FIG. 9 are performed, in a digital medium environment for modeling and selecting digital policies, as part of a computer-implemented method for determining digital policy parameters. In some instances, a non-transitory computer-readable medium stores instructions thereon that, when executed by at least one processor, cause a computing device to perform the acts of FIG. 9. In some implementations, a system performs the acts of FIG. 9. For example, in one or more cases, a system includes one or more memory devices comprising a digital decision model, an importance sampling estimator, a forecasting model, and historical performance metrics of a first set of policies comprising a first set of policy parameters executed by the digital decision model for a set of previous decision episodes. The system further includes one or more server devices configured to cause the system to perform the acts of FIG. 9.

The series of acts 900 includes an act 902 of determining historical performance metrics of a first set of policies. For example, in one or more embodiments, the act 902 involves determining historical performance metrics of a first set of policies applied to a set of previous decision episodes. In some embodiments, the policy parameter generation system 106 determines historical performance metrics of a first set of policies executed by a digital decision model for a set of previous decision episodes.

In at least one implementation, the policy parameter generation system 106 determines the historical performance metrics of the first set of policies applied to the set of previous decision episodes by determining plurality of Markov Decision Process rewards resulting from execution of the first set of policies during the set of previous decision episodes.

In one or more embodiments, the policy parameter generation system 106 determines the historical performance metrics of the first set of policies by: determining a set of states associated with the first set of policies during the set of previous decision episodes; determining a set of actions selected by the first set of policies during the set of previous decision episodes; generating probabilities associated with the first set of policies for selecting the set of actions; and determining policy rewards resulting from selecting the set of actions.

The series of acts 900 also includes an act 904 of determining counter-factual historical performance metrics for a target policy. To illustrate, in some instances, the act 904 involves determining, utilizing the historical performance metrics, a plurality of counter-factual historical performance metrics reflecting application of a target policy having a target policy parameter to the set of previous decision episodes.

In one or more embodiments, determining the plurality of counter-factual historical performance metrics includes determining, utilizing the historical performance metrics, a plurality of reward weights, each reward weight reflecting a comparison between a first performance impact of an action selected using the target policy while in a state and a second performance impact of the action selected using a policy from the first set of policies while in the state; and determining the plurality of counter-factual historical performance metrics based on the plurality of reward weights.

In some cases, the policy parameter generation system 106 processes the historical performance metrics of the first set of policies utilizing an importance sampling estimator to determine a plurality of counter-factual historical performance metrics reflecting application of a target policy having a target policy parameter to the set of previous decision episodes. In some implementations, the policy parameter generation system 106 processes the historical performance metrics of the first set of policies utilizing the importance sampling estimator to determine the plurality of counter-factual historical performance metrics by: processing the historical performance metrics to determine a plurality of reward weights reflecting comparisons between performance impacts of actions selected using the target policy and performance impacts of the actions selected using the first set of policies; and determining the plurality of counter-factual historical performance metrics based on the plurality of reward weights.

Additionally, the series of acts 900 includes an act 906 of generating a forecasted performance metric. For example, in some implementations, the act 906 involves generating a forecasted performance metric for one or more future decision episodes utilizing the plurality of counter-factual historical performance metrics. In some cases, the policy parameter generation system 106 generates, utilizing a forecasting model, a forecasted performance metric for one or more future decision episodes to be executed by the digital decision model by processing the plurality of counter-factual historical performance metrics.

In one or more embodiments, generating the forecasted performance metric for the one or more future decision episodes based on the plurality of counter-factual historical performance metrics includes generating the forecasted performance metric based on a performance trend of the counter-factual historical performance metrics across the set of previous decision episodes. To illustrate, in some instances, the policy parameter generation system 106 generates, utilizing the forecasting model, the forecasted performance metric for the one or more future decision episodes to be executed by the digital decision model by processing the plurality of counter-factual historical performance metrics by utilizing the forecasting model to generate the forecasted performance metric based on a performance trend of the counter-factual historical performance metrics across the set of previous decision episodes.

In some embodiments, the policy parameter generation system 106 generates the forecasted performance metric for the one or more future decision episodes utilizing the plurality of counter-factual historical performance metrics by generating the forecasted performance metric utilizing at least one of an identity-based forecasting model or a fourier-based forecasting model to process the plurality of counter-factual historical performance metrics.

In at least one implementation, the policy parameter generation system 106 generates the forecasted performance metric for the one or more future decision episodes by generating a forecasted Markov Decision Process reward resulting from execution of the target policy during the one or more future decision episodes.

Further, the series of acts 900 includes an act 908 of determining a performance gradient of the forecasted performance metric. For instance, in some cases, the act 908 involves determining a performance gradient of the forecasted performance metric with respect to varying the target policy parameter. For example, in some instances, the policy parameter generation system 106 determines a performance gradient of the forecasted performance metric based on changes to the forecasted performance metric and changes to the plurality of counter-factual historical performance metrics with respect to varying the target policy parameter.

To illustrate, in some embodiments, determining the performance gradient of the forecasted performance metric with respect to varying the target policy parameter includes determining changes to the plurality of counter-factual historical performance metrics with respect to varying the target policy parameter; and determining changes to the forecasted performance metric with respect to the changes to the plurality of counter-factual historical performance metrics. In some implementations, determining the performance gradient of the forecasted performance metric with respect to varying the target policy parameter further includes combining the changes to the plurality of counter-factual historical performance metrics with respect to varying the target policy parameter and the changes to the forecasted performance metric with respect to the changes to the plurality of counter-factual historical performance metrics.

The series of acts 900 also includes an act 910 of modifying a target policy parameter of the target policy. For example, in some instances, the act 910 involves modifying the target policy parameter of the target policy utilizing the performance gradient of the forecasted performance metric. In some cases, the policy parameter generation system 106 modifies, utilizing the performance gradient of the forecasted performance metric, the target policy parameter of the target policy for execution by the digital decision model.

In one or more embodiments, modifying the target policy parameter of the target policy includes modifying the target policy parameter of the target policy to improve an average performance metric for the target policy across the one or more future decision episodes. Indeed, in some embodiments, the policy parameter generation system 106 modifies the target policy parameter of the target policy to improve an average performance metric for the target policy across a plurality of future decision episodes to be executed by the digital decision model. To illustrate, in one or more embodiments, the policy parameter generation system 106 determines a time duration for executing a given policy utilizing the digital decision model, the time duration corresponding to a length of time for executing the plurality of future decision episodes; and modifies the target policy parameter of the target policy to improve the average performance metric for the target policy across the plurality of future decision episodes within the time duration.

In one or more embodiments, the policy parameter generation system 106 determines an entropy regularizer value corresponding to a noise component associated with the one or more future decision episodes; and modifies the target policy parameter of the target policy based on the performance gradient of the forecasted performance metric and the entropy regularizer value.

In some implementations, the series of acts 900 includes acts for changing (e.g., further modifying) the target policy parameter. For example, in some implementations, the acts include determining, utilizing the historical performance metrics, an additional plurality of counter-factual historical performance metrics reflecting application of the modified target policy to the set of previous decision episodes; generating an additional forecasted performance metric for the one or more future decision episodes utilizing the additional plurality of counter-factual historical performance metrics; and changing the modified target policy parameter utilizing an additional performance gradient of the additional forecasted performance metric.

In one or more embodiments, the series of acts 900 further includes acts for executing policies. For example, in some implementations, the acts include executing the target policy with the target policy parameter (e.g., the modified target policy parameter) for the one or more future decision episodes using the digital decision model. In some implementations, executing the target policy with the target policy parameter for the one or more future decision episodes using the digital decision model comprises executing the target policy with the target policy parameter to select a set of actions in at least one Markov Decision Process corresponding to the one or more future decision episodes.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (“SSDs”) (e.g., based on RAM), Flash memory, phase-change memory (“PCM”), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a “NIC”), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed by a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multiprocessor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (“SaaS”), Platform as a Service (“PaaS”), and Infrastructure as a Service (“IaaS”). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 10 illustrates a block diagram of an example computing device 1000 that may be configured to perform one or more of the processes described above. One will appreciate that one or more computing devices, such as the computing device 1000 may represent the computing devices described above (e.g., the server(s) 102, the client devices 110a-110n, and/or the third-party server 114). In one or more embodiments, the computing device 1000 may be a mobile device (e.g., a mobile telephone, a smartphone, a PDA, a tablet, a laptop, a camera, a tracker, a watch, a wearable device). In some embodiments, the computing device 1000 may be a non-mobile device (e.g., a desktop computer or another type of client device). Further, the computing device 1000 may be a server device that includes cloud-based processing and storage capabilities.

As shown in FIG. 10, the computing device 1000 can include one or more processor(s) 1002, memory 1004, a storage device 1006, input/output interfaces 1008 (or “I/O interfaces 1008”), and a communication interface 1010, which may be communicatively coupled by way of a communication infrastructure (e.g., bus 1012). While the computing device 1000 is shown in FIG. 10, the components illustrated in FIG. 10 are not intended to be limiting. Additional or alternative components may be used in other embodiments. Furthermore, in certain embodiments, the computing device 1000 includes fewer components than those shown in FIG. 10. Components of the computing device 1000 shown in FIG. 10 will now be described in additional detail.

In particular embodiments, the processor(s) 1002 includes hardware for executing instructions, such as those making up a computer program. As an example, and not by way of limitation, to execute instructions, the processor(s) 1002 may retrieve (or fetch) the instructions from an internal register, an internal cache, memory 1004, or a storage device 1006 and decode and execute them.

The computing device 1000 includes memory 1004, which is coupled to the processor(s) 1002. The memory 1004 may be used for storing data, metadata, and programs for execution by the processor(s). The memory 1004 may include one or more of volatile and non-volatile memories, such as Random-Access Memory (“RAM”), Read-Only Memory (“ROM”), a solid-state disk (“SSD”), Flash, Phase Change Memory (“PCM”), or other types of data storage. The memory 1004 may be internal or distributed memory.

The computing device 1000 includes a storage device 1006 including storage for storing data or instructions. As an example, and not by way of limitation, the storage device 1006 can include a non-transitory storage medium described above. The storage device 1006 may include a hard disk drive (HDD), flash memory, a Universal Serial Bus (USB) drive or a combination these or other storage devices.

As shown, the computing device 1000 includes one or more I/O interfaces 1008, which are provided to allow a user to provide input to (such as user strokes), receive output from, and otherwise transfer data to and from the computing device 1000. These I/O interfaces 1008 may include a mouse, keypad or a keyboard, a touch screen, camera, optical scanner, network interface, modem, other known I/O devices or a combination of such I/O interfaces 1008. The touch screen may be activated with a stylus or a finger.

The I/O interfaces 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O interfaces 1008 are configured to provide graphical data to a display for presentation to a user. The graphical data may be representative of one or more graphical user interfaces and/or any other graphical content as may serve a particular implementation.

The computing device 1000 can further include a communication interface 1010. The communication interface 1010 can include hardware, software, or both. The communication interface 1010 provides one or more interfaces for communication (such as, for example, packet-based communication) between the computing device and one or more other computing devices or one or more networks. As an example, and not by way of limitation, communication interface 1010 may include a network interface controller (NIC) or network adapter for communicating with an Ethernet or other wire-based network or a wireless NIC (WNIC) or wireless adapter for communicating with a wireless network, such as a WI-FI. The computing device 1000 can further include a bus 1012. The bus 1012 can include hardware, software, or both that connects components of computing device 1000 to each other.

In the foregoing specification, the invention has been described with reference to specific example embodiments thereof. Various embodiments and aspects of the invention(s) are described with reference to details discussed herein, and the accompanying drawings illustrate the various embodiments. The description above and drawings are illustrative of the invention and are not to be construed as limiting the invention. Numerous specific details are described to provide a thorough understanding of various embodiments of the present invention.

The present invention may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

What is claimed is:

1. A non-transitory computer-readable medium storing instructions thereon that, when executed by at least one processor, cause a computing device to:
determine historical performance metrics of a first set of policies applied to a set of previous decision episodes;
determine, utilizing the historical performance metrics, a plurality of counter-factual historical performance metrics reflecting application of a target policy having a target policy parameter to the set of previous decision episodes;
generate a forecasted performance metric for one or more future decision episodes utilizing the plurality of counter-factual historical performance metrics;
determine a performance gradient of the forecasted performance metric with respect to varying the target policy parameter; and
modify the target policy parameter of the target policy utilizing the performance gradient of the forecasted performance metric.

2. The non-transitory computer-readable medium of claim 1, further comprising instructions that, when executed by the at least one processor, cause the computing device to determine the plurality of counter-factual historical performance metrics by:
determining, utilizing the historical performance metrics, a plurality of reward weights, each reward weight reflecting a comparison between a first performance impact of an action selected using the target policy while in a state and a second performance impact of the action selected using a policy from the first set of policies while in the state; and
determining the plurality of counter-factual historical performance metrics based on the plurality of reward weights.

3. The non-transitory computer-readable medium of claim 1, further comprising instructions that, when executed by the at least one processor, cause the computing device to generate the forecasted performance metric for the one or more future decision episodes based on the plurality of counter-factual historical performance metrics by generating the forecasted performance metric based on a performance trend of the counter-factual historical performance metrics across the set of previous decision episodes.

4. The non-transitory computer-readable medium of claim 1, further comprising instructions that, when executed by the at least one processor, cause the computing device to determine the performance gradient of the forecasted performance metric with respect to varying the target policy parameter by:
determining changes to the plurality of counter-factual historical performance metrics with respect to varying the target policy parameter; and
determining changes to the forecasted performance metric with respect to the changes to the plurality of counter-factual historical performance metrics.

5. The non-transitory computer-readable medium of claim 4, further comprising instructions that, when executed by the at least one processor, cause the computing device to determine the performance gradient of the forecasted performance metric with respect to varying the target policy parameter by combining the changes to the plurality of counter-factual historical performance metrics with respect to varying the target policy parameter and the changes to the forecasted performance metric with respect to the changes to the plurality of counter-factual historical performance metrics.

6. The non-transitory computer-readable medium of claim 1, further comprising instructions that, when executed by the at least one processor, cause the computing device to modify the target policy parameter of the target policy by modifying the target policy parameter of the target policy to improve an average performance metric for the target policy across the one or more future decision episodes.

7. The non-transitory computer-readable medium of claim 1, further comprising instructions that, when executed by the at least one processor, cause the computing device to execute the target policy with the modified target policy parameter for the one or more future decision episodes using a digital decision model.

8. The non-transitory computer-readable medium of claim 1, further comprising instructions that, when executed by the at least one processor, cause the computing device to:
determine the historical performance metrics of the first set of policies applied to the set of previous decision episodes by determining plurality of Markov Decision Process rewards resulting from execution of the first set of policies during the set of previous decision episodes; and
generate the forecasted performance metric for the one or more future decision episodes by generating a forecasted Markov Decision Process reward resulting from execution of the target policy during the one or more future decision episodes.

9. The non-transitory computer-readable medium of claim 1, further comprising instructions that, when executed by the at least one processor, cause the computing device to generate the forecasted performance metric for the one or more future decision episodes utilizing the plurality of counter-factual historical performance metrics by generating the forecasted performance metric utilizing at least one of an identity-based forecasting model or a fourier-based forecasting model to process the plurality of counter-factual historical performance metrics.

10. The non-transitory computer-readable medium of claim 1, further comprising instructions that, when executed by the at least one processor, cause the computing device to:
determine, utilizing the historical performance metrics, an additional plurality of counter-factual historical performance metrics reflecting application of the modified target policy to the set of previous decision episodes;
generate an additional forecasted performance metric for the one or more future decision episodes utilizing the additional plurality of counter-factual historical performance metrics; and
change the modified target policy parameter utilizing an additional performance gradient of the additional forecasted performance metric.

11. A system comprising:
one or more memory devices comprising a digital decision model, an importance sampling estimator, a forecasting model, and historical performance metrics of a first set of policies comprising a first set of policy parameters executed by the digital decision model for a set of previous decision episodes; and
one or more server devices configured to cause the system to:
process the historical performance metrics of the first set of policies utilizing the importance sampling estimator to determine a plurality of counter-factual historical performance metrics reflecting application of a target policy having a target policy parameter to the set of previous decision episodes;
generate, utilizing the forecasting model, a forecasted performance metric for one or more future decision episodes to be executed by the digital decision model by processing the plurality of counter-factual historical performance metrics;
determine a performance gradient of the forecasted performance metric based on changes to the forecasted performance metric and changes to the plurality of counter-factual historical performance metrics with respect to varying the target policy parameter; and
modify, utilizing the performance gradient of the forecasted performance metric, the target policy parameter of the target policy for execution by the digital decision model.

12. The system of claim 11, wherein the one or more server devices are configured to cause the system to determine the historical performance metrics of the first set of policies by:
determining a set of states associated with the first set of policies during the set of previous decision episodes;
determining a set of actions selected by the first set of policies during the set of previous decision episodes;
generating probabilities associated with the first set of policies for selecting the set of actions; and
determining policy rewards resulting from selecting the set of actions.

13. The system of claim 11, wherein the one or more server devices are further configured to cause the system to:
determine an entropy regularizer value corresponding to a noise component associated with the one or more future decision episodes; and
modify the target policy parameter of the target policy based on the performance gradient of the forecasted performance metric and the entropy regularizer value.

14. The system of claim 11, wherein the one or more server devices are configured to cause the system to process the historical performance metrics of the first set of policies utilizing the importance sampling estimator to determine the plurality of counter-factual historical performance metrics by:
processing the historical performance metrics to determine a plurality of reward weights reflecting comparisons between performance impacts of actions selected using the target policy and performance impacts of the actions selected using the first set of policies; and
determining the plurality of counter-factual historical performance metrics based on the plurality of reward weights.

15. The system of claim 11, wherein the one or more server devices are configured to cause the system to modify the target policy parameter of the target policy for execution by the digital decision model by modifying the target policy parameter of the target policy to improve an average performance metric for the target policy across a plurality of future decision episodes to be executed by the digital decision model.

16. The system of claim 15, wherein the one or more server devices are further configured to cause the system to:
determine a time duration for executing a given policy utilizing the digital decision model, the time duration corresponding to a length of time for executing the plurality of future decision episodes; and
modify the target policy parameter of the target policy to improve the average performance metric for the target policy across the plurality of future decision episodes within the time duration.

17. The system of claim 11, wherein the one or more server devices are configured to cause the system to generate, utilizing the forecasting model, the forecasted performance metric for the one or more future decision episodes to be executed by the digital decision model by processing the plurality of counter-factual historical performance metrics by utilizing the forecasting model to generate the forecasted performance metric based on a performance trend of the counter-factual historical performance metrics across the set of previous decision episodes.

18. In a digital medium environment for modeling and selecting digital policies, a computer-implemented method for determining digital policy parameters comprising:
determining historical performance metrics of a first set of policies executed by a digital decision model for a set of previous decision episodes;
performing a step for determining a target policy parameter for a target policy for one or more future decision episodes to be executed by the digital decision model from the historical performance metrics of the first set of policies for the set of previous decision episodes; and
executing the target policy with the target policy parameter for the one or more future decision episodes using the digital decision model.

19. The computer-implemented method of claim 18, wherein identifying the historical performance metrics of the first set of policies executed by the digital decision model for the set of previous decision episodes comprises:
determining a set of states associated with the first set of policies during the set of previous decision episodes;
determining a set of actions selected by the first set of policies during the set of previous decision episodes;
generating probabilities associated with the first set of policies for selecting the set of actions; and
determining policy rewards resulting from selecting the set of actions.

20. The computer-implemented method of claim 18, wherein executing the target policy with the target policy parameter for the one or more future decision episodes using the digital decision model comprises executing the target policy with the target policy parameter to select a set of actions in at least one Markov Decision Process corresponding to the one or more future decision episodes.
